(19) United States
(12) Patent Application Publication
(10) Pub. No.: US 2007/0037185 A1
(43) Pub. Date: Feb. 15, 2007

(54) Estimating allele frequencies by small pool PCR

(75) Inventors: Mary Coolbaugh-Murphy, Houston, TX (US); Barry W. Brown, Houston, TX (US); Louis Ramagli, Missouri City, TX (US); Michael J. Siciliano, Houston, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/432,732

(22) Filed: May 11, 2006

Related U.S. Application Data
(60) Provisional application No. 60/679,895, filed on May 11, 2005.
(60) Provisional application No. 60/682,155, filed on May 18, 2005.

(51) Int. Cl. C12Q 1/68; G06F 19/00

(52) U.S. Cl. 435/6; 702/20

(57) ABSTRACT

Methods of the invention include the application of fluorescent technology, total genome amplification, high throughput automated microsatellite fragment analysis, robotics, and novel computational methods. Computational methods include determining a microsatellite instability (MSI) phenotype (frequency and significance of MSI over multiple loci) using SP-PCR at higher than 0.5 genome equivalents (0.5 to 2 genome equivalents).

This application claims priority to U.S. Provisional Patent applications Ser. No. 60/679,895 filed on May 11, 2005, and Ser. No. 60/682,155 filed May 18, 2005, entitled “ESTIMATING ALLELE FREQUENCIES BY SMALL POOL PCR,” each of which is incorporated herein by reference in its entirety.

The United States Government may own rights in the present invention pursuant to grant CA34936, CA95567, and CA112508 from the United States National Institutes of Health.

### BACKGROUND OF THE INVENTION

I. Field of the Invention

Embodiments of the invention are related to molecular genetics, genomics, and oncology. Particular embodiments are related to genomic small pool PCR and its use in genomic analysis, diagnosis, and cancer surveillance methodologies.

II. Background

While scanning the genomes of tumor DNA from hereditary non-polyposis colon cancer (HNPCC) patients by PCR using polymorphic microsatellite loci for detecting loss of heterozygosity (LOH) (Aaltonen et al., 1993; Ionov et al., 1993; Thibodeau et al., 1998), a remarkable observation was made—the presence of new microsatellite alleles (different fragment sizes) in addition to the progenitor alleles with which the patients were born. It soon was determined that the enabling events giving rise to such phenotypes occurred when mismatch repair (MMR) genes were either mutated (Fishel et al., 1993) or silenced (Kane et al., 1997). It was hypothesized that such events should have severe clinical consequences in that the inability to repair replication errors could result in accelerated tumor initiation and progression based upon the observation that HNPCC patients present with disease symptoms 20 years earlier than the general population (Lynch, 1993).

For this microsatellite instability (MSI) to be detected by simple PCR against a background of progenitor fragments, mutant fragments must be present at a frequency >0.25. Recommendations (Boland et al., 1998) that have been widely implemented to evaluate MSI levels in HNPCC involve study of at least five of several recommended microsatellite loci and if new fragments were seen in at least 2 (or 40%) of those loci, the sample was considered MSI-H (high) whereas failing to achieve that, tumors were grouped together into a MSI-L (low, where mutant fragments were observed at only one locus of the five) or MSS (stable, no mutant fragments seen at any of the loci screened) class. Though not statistically rigorous, this categorization has proven useful as the MSI-H phenotype has come to be recognized as a distinct class resulting from serious mutations or expression changes in at least one of the major mismatch repair genes, MSH2 or MLH1 (Jass, 1999). However, this approach to quantification gives no information on the frequency of mutant fragments at loci screened. Indeed, if the minimum frequency of mutant fragments observable is 0.25, it is possible that lower, yet possibly clinically significant levels, of MSI may play a role in carcinogenesis. Therefore the ability to see and quantify MSI at such levels is indicated.

Both the mutant frequency and the sensitivity of detection issues can be addressed by employing small pool PCR (SP-PCR) (Monckton and Jeffreys, 1991). There the DNA from the tissue being studied is diluted so that the amount used for PCR contains only approximately a single diploid genome equivalent (g.e.) of DNA. PCR is then conducted on multiple (approximately 100) such small pools so that if the frequency of mutant fragments is over 1% there is a high probability of trapping such fragments in some of the small pools. Such fragments within such small pools are then no longer “overwhelmed” by the presence of the more frequent progenitors and can be identified and counted after amplification. Interestingly, the concept was applied to HNPCC (Parsons et al., 1995) almost 10 years ago with a remarkable result—detection of MSI in the constitutive (non-tumor) tissue of patients carrying germ line mutations in MMR genes. This finding has possible consequences for understanding inherited cancer and identification of individuals at risk, has had very little follow up—possibly because the procedure is extremely labor intensive with a great potential for artifact, contamination, and operator error leading to false positive results.

There remains a need for methods that are robust and reliable enough for the accurate and specific determination of allele frequencies and/or MSI in a sample.

### SUMMARY OF THE INVENTION

The inventors have developed procedures for single molecule PCR and adapted methods for increasing throughput—using fluorescently labeled probes and multiplexing loci for resolution and detection using automated fragment analysis apparatus and software (Canzian et al., 1996)—and maintaining quality control (Zhang et al., 1994). Here the inventors have combined those methods with a new statistical approach to determine frequencies, and significance between frequencies, of Poisson distributed data. Also total genome amplification procedures have been developed to operate at single genome levels. Additional validation experiments and robotic technology to protect against error and contamination providing a robust methodology for assessing MSI has been put in place.

As described herein, the inventors have developed a methodology to detect the frequency of alleles, including mutant alleles, at multiple genetic loci, e.g., multiple microsatellite loci, in tissues or samples from human beings and other animals. The samples can be obtained by various minimally invasive methods. This makes it possible to establish a MSI phenotype as a measure of cancer risk in individuals. Previous art diluted DNA to less than 0.5 genome equivalents and then conducted PCR to amplify alleles at specific microsatellite locus in many pools of such DNA—small pool PCR or SP-PCR. This enabled the detection of mutant alleles, which were present in the original DNA at frequencies as low as 1%, but was time consuming and lacked reliability. The current methods include the application of fluorescent technology, total genome amplification, high throughput automated microsatellite fragment analysis, and robotics, as well as novel computational methods. Of particular significance, new statistical methods for determining an MSI phenotype (frequency and significance of MSI over multiple loci) by SP-PCR at higher than 0.5 genome equivalents (0.5 to 2 genome equivalents) makes the procedure practical for measuring such levels of MSI as an indicator of cancer risk.

Embodiments of the invention include methods for genomic analysis comprising the steps of: (a) obtaining DNA from a sample; (b) diluting the DNA to less than 10 genome equivalents of DNA; (c) performing a plurality of amplifications on the diluted DNA using amplification primers for a plurality of genetic markers; (d) calculating allele frequency of the genetic markers amplified; and (e) assessing significance of the allele frequency within a sample or between two or more samples. Further steps can include performing whole genome amplification on the DNA dilution of step (b) prior to step (c). In certain aspects, the DNA is diluted to 0.5, 0.75, 1.0, 1.25, 1.5, 1.75 to 2, 2.25, 2.5, 2.75, 3, genome equivalents including all values and ranges there between, prior to step (c). Calculating an allele frequency typically comprises: (a) determining a maximum likelihood estimate of the mean number of alleles for each amplification; and (b) determining a frequency for each allele (allele frequency) across all amplifications of a DNA sample. Typically, at least one allele is a mutant allele or an allele of interest. In further aspects of the invention a mutant allele frequency is less than 0.1, 0.15, 0.20 or 0.25. The mutant frequency can also be in the range of 0.001, 0.005, 0.01, 0.05 to 0.15, 0.20 0.25, including all values and ranges there between.

Embodiments of the invention also include methods for assessing an allele frequency in a DNA sample comprising the steps of: (a) amplifying the DNA of the sample using amplification primers for at least one genetic marker; and (b) calculating an allele frequency (fᵢ) of the amplified genetic markers. The allele frequency can be determined by the formula:

$${\hat{f}}_{i} = \frac{{\hat{μ}}_{i}}{\hat{c}} ,$$

wherein \hat{μ}ᵢ is the maximum likelihood estimate of the mean number of allele i; and ĉ is the estimate of the calibration quantity.

Aspects of the invention include methods for determing the total mutant frequency m as determined by the formula:

$$\hat{m} = \frac{{\sum }_{k} {\hat{μ}}_{k}}{{\sum }_{j} {\hat{μ}}_{j}}$$

where μ is the maximum likelihood estimate of the mean number of mutant alleles k and all alleles j; and j ranges over all alleles and k ranges over all mutant alleles.

Aspects of the invention include the analysis of genetic markers. Genetic marker include, but are not limited to, site specific markers, multilocus markers, or a combination of site specific and multilocus markers. A genetic marker is typically a variable number tandem repeat (VNTR) marker, a minisatellite marker, a microsatellite marker, or a single nucleotide polymorphism (SNP) marker.

A further aspect of the invention includes the analysis of a variety of samples. For example, DNA can be isolated from a cell, a tissue, a forensic sample, or a biological fluid. Typically, DNA is isolated from a blood sample, a buccal wash, a buccal swab, a vaginal swab, a histopathological sample, a skin sample, a skin scrape, sloughed skin, a biopsy, urine, saliva, semen, or a hair follicle.

In still further aspects, the methods provide for amplification of small quantities of DNA. Amplification can be performed on 0.5, 0.75, 1, 1.25, 1.5 to 2 genome equivalents of DNA, or 1, 2, 3, 4, 5, 6, 7, 8, 9, 10, 11 to 12 pg of DNA, including all values and ranges there between.

Aspects of the invention include genomic assessment of a subject having a disease or pathological condition. A sample can be obtained from a subject that has, is suspected of having, or is at risk for developing cancer or a hyperproliferative condition. A subject may be undergoing cancer therapy. In certain aspects, an allele or a mutant frequency is correlated to development of resistance to a cancer therapy, risk of further progression, or aggressiveness of disease. In certain aspects a subject is a member of a family with a history of cancer, and/or has been exposed or is suspected of being exposed to a genotoxic substance or environment. The method typically will correlate the allele frequencies, e.g., a mutant allele frequency, to a disease or condition, e.g., predisposition for cancer. A subject that presents a correlation indicating a pre-disposition for a disease will typically be monitored more frequently or more closely development of a disease state. In the instance of a subject predisposed for cancer monitoring will be increased for cancerous lesions or the subject will be administered cancer preventative treatments.

Further embodiments include methods of reconstructing the genotype of a subject comprising the steps of: (a) obtaining DNA from a subject with an unknown genotype or haplotype; (b) conducting a plurality of SP-PCR on the DNA amplifying a plurality of genetically linked markers; (c) assessing the phase of genetic markers by analysis of concordant amplification of genetically linked markers; and (d) reconstructing a genotype or haplotype based on phase of genetically linked markers.

Still further embodiments include methods of genotyping a subject comprising the steps of: (a) obtaining DNA from a subject with an unknown genotype; (b) diluting the DNA to obtain a DNA dilution comprising 0.5 to 2 genome equivalents of DNA; (c) conducting a plurality of SP-PCR on the DNA amplifying a plurality of genetic markers; (d) assessing the amplification of genetic markers; and (e) determining the linkage of the genetic markers to a trait or marker based on the assessment of the SP-PCR amplifications. The methods typically include assessment of the genetic markers comprising (a) determining a maximum likelihood estimate of the mean number of alleles for a genetic marker in each amplification; and (b) determining a frequency for each allele (allele frequency) across all amplifications for a DNA sample. The method can further include performing whole genome amplification on the DNA dilution of step (b) and using the amplified DNA for step (c). The whole genome amplification can be performed on 0.5 to 2 genome equivalents of DNA.

Procedures are described that apply automated analyses and robotics for: multiplexing of the products of multiple microsatellite loci after SP-PCR; increasing the speed and accuracy of reagent distribution; reducing possibilities of contamination; and making it possible to determine mutant frequencies without the need to reduce the amount of DNA in the small pool reactions to <0.5 genome equivalents. This latter capability plus identification of a smaller set of loci informative for MSI, greatly reduces the time and effort of the analysis. The system has been tested to quantify what one might consider the most subtle of increases in a MSI phenotype—increases with age in normal tissue in normal blood bank volunteers (e.g., peripheral blood lymphocytes or PBLs). These studies have been successful and have provided an additional statistical tool for evaluating increased MSI phenotype levels in PBLs and epithelial cells present in saliva of individuals with a genetic predisposition to cancer. More importantly, it established that the technological innovations would make the MSI phenotype analysis available to determine genetic risk for cancer. The system does indeed detect significantly higher MSI in the PBLs of patients with known hereditary predispositions to cancer.

One important aspect of the invention includes the protection against false positive results in determining the mutant frequencies. Since the 1 to 2 g.e. of DNA in each small pool results in the data in any experiment fitting a Poisson distribution, it has become necessary to develop a statistical approach for determining mutant frequencies and for calculating the significance of differences between frequencies.

These methods are exemplified by assessing material from two colon cancer patients with high levels of MSI in their tumor tissues. The data so generated determine the frequency of mutant alleles in a tumor and adds to the observation that constitutive tissue from a patient bearing a germ line MMR mutation has detectable MSI. Comparisons of the statistical methods employed with other methods that have been used and significance of MSI obtained are elaborated upon. A computer program for the described calculations has also been developed.

Microsatellite instability (MSI) by can be identified by partitioning DNA into multiple small pools containing only single genome amounts of DNA. Amplification of these pools results in trapping of both progenitor and low frequency mutant alleles where they can be identified and quantitated. Statistical approaches determining both the frequencies, and significant differences between frequencies, of these Poisson-distributed alleles are presented. Results indicate a level of sensitivity and quantification not possible by standard PCR methods. Using material from colon cancer patients with high levels of MSI in their tumors, the molecular and robotic methods for carrying out such studies are exemplified. Validation experiments indicated mutants are detectable at frequencies of above background of >0.03 and lower. Frequencies, obtained in tumor tissue (>0.25), met the expectations of the approach. Significant levels of MSI were detected in the constitutive tissue of the patient carrying a germ line mutation for mismatch repair suggesting both mechanistic and clinical applications of the procedure.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Chromatograms showing fluorescently labeled PCR products of the microsatellite locus, DMPK. Sizes (number of repeats) of progenitor alleles are labeled as 5 repeats and 20 repeats. (FIG. 1A) Tissue is the dissected normal colon from the MSI-H HNPCC patient. Top panel had over 100 genome equivalents (g.e.) of DNA amplified and indicates the sample is from a heterozygous individual at this locus—5 repeats and 20 repeats. Those peaks are clear and have the ever present smaller “stutter” bands. The bottom two panels are two of the many small pools (<2 g.e.). Most pools had either one, the other, both (as in the middle panel), or no progenitor fragments. In the bottom panel, in addition to the two progenitor fragments, a mutant fragment (19 repeats) at the size of the stutter band from the progenitor 20 repeat fragment is visible. (FIG. 1B) Tissue is colon tumor from the same patient. The top panel is a traditional PCR showing the progenitor fragments (5 and 20 repeats). In this case, the 19 repeat mutant is present in such high frequency as to be visible by traditional PCR. The bottom three panels are selected small pools (2 g.e.) where mutant fragments (17 and 21 repeats) are visible in addition to the common 19 repeat mutant and the progenitor fragments. The bottom panel shows that the 20 progenitor fragment need not be present for the mutant 19 fragment to be seen.

FIGS. 2A-2B. Distribution of estimates of the mutation frequency in 1000 random replicates (FIG. 2A). Distribution after applying the arcsin transformation (FIG. 2B).

FIG. 3. Diagrammatic illustration of the general mechanism of SP-PCR and how it increases detection of rare events.

FIG. 4 Illustrates a general overview of SP-PCR and hemi-nested PCR of a genetic marker.

FIGS. 5A-5C. Shows representative chromatograms of small pools of the 3 microsatellite loci. Samples used were heterozygous for D2S123 (FIG. 5A) and D5S346 (FIG. 5B) and homozygous for D17S518 (FIG. 5C). Vertical lines show positions of progenitor alleles. In some pools both heterozygous progenitor alleles were captured (panel A of D2S123 and D5S346). In some pools no alleles were present (panel B of D2S123 and D5S346). Individual progenitor alleles were segregated (panels D and E of D2S123 and D5S346). Mutant alleles were captured either alone (panels C of D2S123, B of D17S518) or with a progenitor allele (panel C of D5S346 and D17S518).

FIG. 6. Summarizes data plotted against MSI frequency in the PBL DNA of normal individuals at various ages. Normal controls are squares. Circles are the 6 HNPCC patients and the diamonds are the sporadic CRC patients.

FIGS. 7A-7F. Shows representative chromatograms of SP-PCR products of six microsatellite loci. Vertical black lines represent the positions of the progenitor alleles for each of the loci of the subjects for this set of data. D17S518 and BAT26 were homozygotes while subjects for the remaining loci were heterozygotes. Size markers are included as non filled peaks. Across the top of each panel for each locus are indicated PCR fragment sizes in number of nucleotide pairs. Peaks shaded in are either primary progenitor or mutant peaks and may overlap stutter bands. At 0.75 genome equivalency, for each locus there are blank lanes, or lanes where progenitor alleles are separated from each other or from mutant alleles. Allele readings: D17S518 (FIG. 7A) A progenitor, B mutant, C mutant and progenitor, D progenitor; D2S123 (FIG. 7B) A both progenitors, B empty well, C mutant, D small progenitor, E large progenitor; BAT26 (FIG. 7C) A progenitor, B empty well, C mutant, D progenitor, E progenitor and smaller mutant; D17S250 (FIG. 7D) A large progenitor, B empty well, C large progenitor and smaller mutant, D small progenitor; D5S346 (FIG. 7E) A both progenitors, B empty well, C mutant smaller that either progenitor and large progenitor, D large progenitor, E small progenitor; DMPK (FIG. 7F) A both progenitors, B empty well, C small progenitor, D small progenitor and very small mutant, E both progenitors and smaller mutant.

FIGS. 8A-8F. Shows MSI data for each of the six loci plotting In (mutant frequency/(1-mutant frequency)) [Logit] against age of each individual. A linear regression line is plotted for each locus and each equation is presented in the upper left corner. For all regression lines are indicated the p-values evaluating the probabilities that the differences between the linear regression lines, and a line with slope equal to zero, are due to chance. D2S123 (FIG. 8A); D17S250 (FIG. 8B); BAT26 (FIG. 8C); D5S346 (FIG. 8D); D17S518 (FIG. 8E); DMPK (FIG. 8F).

FIG. 9. The Logit of the mean average of mutant frequency all 6 loci at each age is plotted simultaneously with the mean average of mutant frequency of three select loci (D2S123, D5S346 and D17S518). The linear regression line (-) for all 6 loci is parallel to the regression line (—) for the select 3 loci. Regression equations and the p-value that states significance from a null hypothesis of no correlation to age [a zero slope line] are listed in the upper left corner.

FIG. 10. General flow diagram for computer implemented analysis of SP-PCR amplifications.

### DETAILED DESCRIPTION OF THE INVENTION

As a result of rapidly developing genomic testing, whole nucleic acid analysis is a task being performed in many genetic laboratories. The polymerase chain reaction (PCR) is a well established method for amplifying nucleic acid sequences, and the method is routinely used in numerous application areas, such as microbiological testing, expression studies, determination of genetic variation in population, genetic testing, forensics, and food and environmental testing. Testing of nucleic acids using PCR generally involves three steps: sample preparation, amplification and detection. However, the processes for performing nucleic acid analysis are often laborious and inefficient. Even the more sensitive PCR assays that are used on lesser genome equivalents are weak and less robust than the present methods.

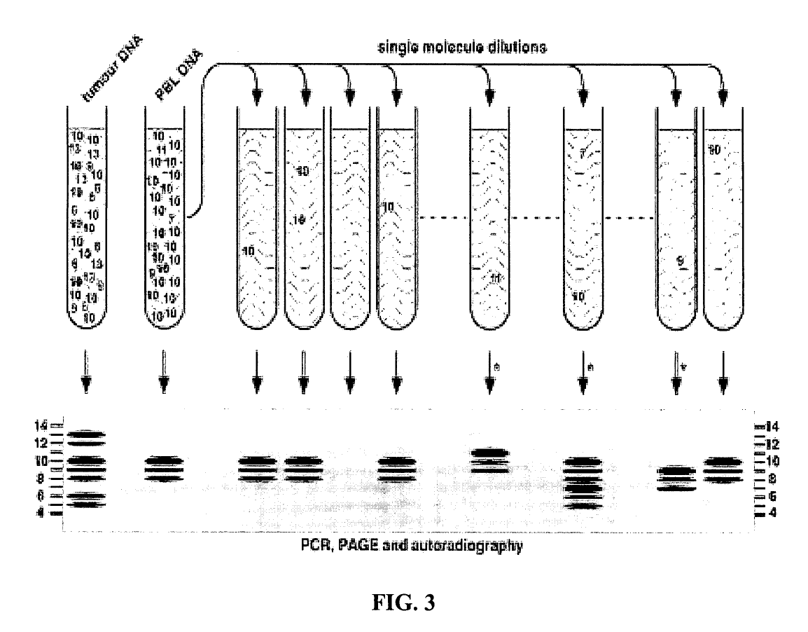

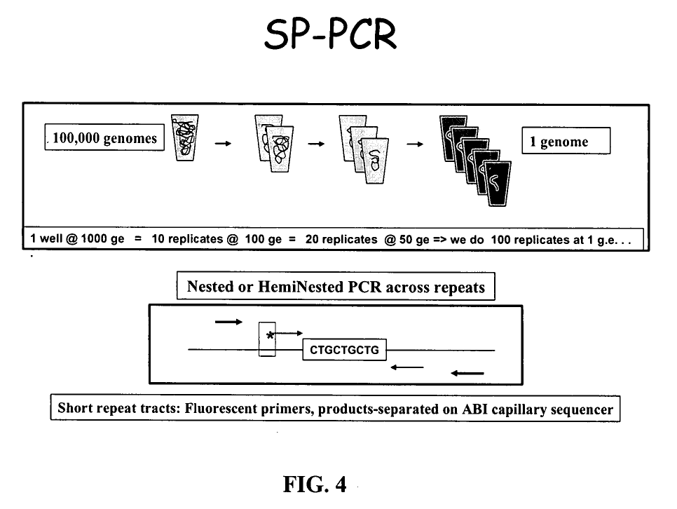

Mutations in DNA can result in the measurable increases of microsatellite instability (MSI) in the DNA of “normal” somatic cells. The present invention provides methods for the identification of a molecular phenotype in normal cells that identify people at increased risk for cancer and other conditions. The methods include small pool PCR (SP-PCR) to quantify MSI, for example in peripheral blood lymphocytes (PBL) DNA, by diluting DNA to about single genome equivalents and conducting microsatellite PCR on over, for example, 100 such small pools so that mutant microsatellite fragments as infrequent as 1% or less can be identified and counted (see FIG. 3 and FIG. 4 for a general overview).

Embodiments of the invention include obtaining samples of DNA from a subject to be assessed for conditions related directly or indirectly with microsatellite instability. Upon receipt of the DNAs, genotyping can be done by standard PCR, e.g., using BAT26; D2S123, D5S346, D17S250, D17S518; and DMPK loci. Multiplexed PCR products can be genotyped on the ABI 3100 (ABI, Foster City, Calif.). Samples can be quantified using a known and characterized locus, such as the beta-globin locus to determine the amount of amplifiable DNA present in the samples. DNAs can then diluted to approximately single diploid genome levels.

The diluted DNA samples are then subjected to small-pool PCR. The product of the SP-PCR methodology is analyzed using apparatus, methods, and computer programs designed for assessing the data using novel statistical methods and determining if a sample indicates the presence, absence, or increase in MSI.

Aspects of the analytical methods include, but are not limited to input of SP-PCR data into a format readable by an analysis program (e.g., computer implementation of the statistical methods described below). Data includes for each well (replicate data item), the amount of DNA in experimenter's units in the well and the identity of the alleles seen in the well. For each allele separately, the quantity μ and μ variance are estimated. The value μ is the average number of alleles per well, and equals the calibration constant (c), estimated later in the process) times the frequency of that particular allele. The asymptotic variance (measure of variation) of μ is also calculated. The calibration constant (c) is the number that multiplies an experimenter's unit to yield an amount of DNA that is an allele equivalent (a.e.) (i.e., an average of one allele per well). It is estimated as the sum over all the different alleles seen of the μ's. The estimate of the frequency of each allele (f) is its μ divided by the calibration constant. The asymptotic variances of the estimates of the mutation frequencies is calculated for each allele. The asymptotic variance is a measure of error for a very large number of wells. Theory doesn't tell us how many wells is sufficient for this approximation to hold. Hence, an alternative estimate of the variance is typically used by estimating the calibration constant and the frequencies of each allele at least or at most a 1000 times. Each step yields a new estimate of the calibration constant and of the frequencies of each allele. These 1000 replicates are used to compute the variance of the calibration constant and of the frequencies of each allele. If the answer so obtained differs from the asymptotic answer, the answer so obtained is preferred.

From the estimates of the calibration constant and the frequencies of each allele, random numbers are used to generate another data set similar, except for random variation, to that read in. The calculation of the calibration constant and the asymptotic variance can be repeated on each generated data set to produce another estimate of the calibration constant and the frequency of each allele.

I. Small Pool Polymerase Chain Reaction (SP-PCR)

Small pool PCR is typically performed using multiple hemi-nested or nested SP-PCRs conducted on DNA samples of interest. A plurality of alleles distributed over a number of PCR replicates per sample can be amplified at each locus. One of skill in the art is able to consult a variety of known public and private databases that contain information regarding genetic markers such as microsatellite repeats or simple sequence repeats (SSR) and determine which primer sets to obtain to amplify any of the repeats in the database. Examples of such databases include, but are not limited to UniSTS database maintained by the United States National Center for Biotechnology Information (NCBI) and Microsatellites Repeats Database (MRD, available on the world wide web at ccmb.res.in/mrd/).

SP-PCR will typically be conducted using automated and semi-automated methods. These methods can use automated sample preparation equipment, robotic sample handling and plating, automated thermocyclers, and/or automated sequence analysis equipment, much of which is commercially available and adaptable to the present methods. In certain embodiments, a MWG Biotech RoboSeq 4204 S™ robot with an onboard Primus-HT 384™ thermocycler (MWG Biotech, High Point, N.C.) can be used for setup and amplification of the initial “outer” PCR and the distribution of the secondary “inner” PCR after which they were diluted, for example, using the Qiagen BioRapidPlate™ and Twister I™ robots (Valencia, Calif.). The terms “outer” and “inner” designate the relative position of the PCR primers used to amplify a target sequence (i.e., an amplicon), thus the outer primers will the primer(s) that hybridize to a sequence the furthest 5′ of an amplicon, whereas the inner primer will hybridize 3′ to the outer primer and whose sequence may overlap with the outer primer sequence. The secondary “inner” PCR (i.e., amplification where at least one primer hybridizes 3′ prime to an outer primer) can be amplified using MWG Biotech Dualblock Primus-HT 384™ thermocycler (High Point, N.C.). The RapidPlate™ can be used to multiplex all the loci's 384-well trays for each sample set, resulting in 384 wells, each containing the SP-PCR products of multiple loci. The trays of multiplexed SP-PCR products can be analyzed on an ABI ₃₁₀₀™ capillary system, running GeneScan™ software. PCR products may be labeled by including various dyes in the amplification reaction or coupled to one or more primers. Dyes include, but are not limited to 6-FAM, NED and VIC on the primers, and ROX for the internal size standard on the ABI. Certain primers and dyes used for exemplary loci were described in Coolbaugh-Murphy et al. (2004), with the exception of D17S518.

DNA samples are quantified initially by UV spectrophotometer and verified at one or more control loci, e.g., D2S123 or markers associated with one or more chromosomal regions. A primary amplification reaction is performed with outer primers using about 0.75 g.e. DNA. DNA samples are arrayed on a plate or similar apparatus having about 110 replicates. The primary reactions are diluted and an aliquot distributed into a volume of master mix (mix containing the general components of an amplification like polymerase, nucleotides, buffer, etc.) containing inner primers for a secondary reaction plate. In certain aspects of the invention the samples are diluted 1:10, 1:20, 1:50, or 1:100 or more. The handling of the amplifications can be programmed and accomplished by using a robotic system such as a Qiagen BioRapidPlate and Twister I robots inside an AirClean Hood to prevent contamination.

A. Hemi-nested SP-PCR.

Methods for amplifying a sample using hemi-nested PCR will typically include one or more of the following steps: quantitation—upon receipt of DNA sample, conduct initial quantitation with UV spectrophotometer; dilution—DNA's may be serially diluted to about 600, 60, and 6 pg/μl concentrations or less; calibrations—calibration includes: (a) conducting hemi-nested (Fluorescently labeled) Small-pool PCR of multiple 3.0-6.0 pg (1-2 alleles) replicates (n=˜32) of given DNA at the reference (D2S123) locus. Multiple samples are PCR'd simultaneously on 384-well plates, and PCR products processed using MWG and Qiagen robotics. (b) PCR products are analyzed, for example, on ABI 3100 capillary electrophoresis machine, using GeneScan™ software to assign product sizes and peak heights. (c) the number of each allele in each well is counted/scoredl, data is entered into the SPPCR v. 1.0 program. (d) the program reports the estimated amount of PCR-able DNA present in the DNA aliquot(s) analyzed; quantitation of allele frequency—quantitation of mutant frequency or other genetic alteration includes: (a) using the calibration data, amplifying˜144 alleles in 112 replicates at each locus for each sample, single molecule (4.5 pg, 0.75 g.e.) level on 384-well plates as above: (b) hemi-nested, fluorescently labeled PCR on multiple 384-well plates, using MWG and Qiagen robotics to process; 1 locus per plate, multiplex labeled PCR products robotically. (c) run multiplexed PCR products on ABI 3100, GeneScan analysis of products. (d) Count number of each allele observed at each locus, both progenitors and variants. (e) Enter data into SPPCR v.1.0 program. (f) Program estimates number of alleles analyzed, mutant frequency, and significance of variance between patient/unknown samples mutant frequencies compared to matched, unrelated normal controls. (g) Determine “MSI-phenotype” based on weighted average of mutant frequencies at multiple loci. (See FIG. 4 for general illustration).

B. Whole Genome SP-PCR

Whole genome amplification (WGA) of small pool levels of DNA (6 pg) enables the molecular haplotyping of a whole series of linked loci on a chromosome as indicated above. Beyond that, it would reduce by ⅓ the amount of amplifiable DNA needed for a SP-PCR analysis of MSI (from <3.0 ng to 0.7 ng) and reduce by ⅓ the amount of time and reagents to conduct a study. GE Healthcare Life Sciences puts out a kit for WGA called GenomiPhi™ which uses the phi29 enzyme for rolling circle amplification of DNA. It is not recommended for DNA quantities of <1 ng. The inventors have modified the recommended procedures in the following manner for WGA of the 6 pg of DNA in our small pools—the length of denaturation time is increased and the total volume of the reaction is reduced to 10 μl. All reactions are typically performed in 384 well plates without oil in a MWG Primus thermocycler. PCR is then conducted on 2 μl from each well at each of three microsatellite loci at which a DNA sample is heterozygous—e.g., D2S123, D5S346, and D17S518. The expected number of fragments have been recovered for each locus and remarkably each allele at each locus was recovered at the same frequency. Therefore, there was no allele dropout with the procedure and WGA was evaluated as perfectly appropriate for the future studies to be performed.

Typically methodology of WGA include: DNA quantitation—upon receipt of DNA sample, conduct initial quantitation with UV spectrophotometer; serial dilution of DNA—serially dilute stock DNA's to 600, 60, and 6 pg/μl concentrations or less; calibration—to calibrate a 6 pg quantitation: (a) achieved by hemi-nested (Fluorescently labeled) small-pool PCR of multiple 3.0-6.0 pg (1-2 alleles) replicates (n=˜32) of given DNA at the reference (D2S123) locus. Multiple samples PCR'd simultaneously on 384-well plates, and PCR products processed using MWG and Qiagen robotics. (b) Analyze PCR products on ABI 3100 capillary electrophoresis machine, using GeneScan software to assign product sizes and peak heights. (c) Count/score the number of each allele seen in each well, enter data into the SPPCR v.1.0 program. (d) Program reports the estimated amount of PCR-able DNA present in the DNA aliquot(s) analyzed; frequency quantitation—Quantitate mutant frequency or other genetic alteration: by (a) using calibration data, amplify ˜144 alleles in 112 replicates by whole genome amplification for each sample, (b) whole genome amplification at single molecule (3 pg, 0.5 g.e.) level on 384-well plates, using MWG and Qiagen robotics to process, then dispense diluted products to multiple 384 well PCR replica plates, at one plate per locus; (c) conduct locus-specific PCR with fluorescently labeled primers, then robotically multiplex labeled PCR products; (d) run multiplexed PCR products on ABI 3100, GeneScan analysis of products; (e) count number of each allele observed, both progenitors and variants; (f) enter data into SPPCR v.1.0 program or similar program; (g) program estimates number of alleles analyzed, mutant frequency, and significance of variance between patient/unknown samples mutant frequencies compared to matched, unrelated normal controls; and (h) Determine “MSI-phenotype” based on weighted average of mutant frequencies at multiple loci.

C. Data Analysis and Statistical Development

Typically, chromatograms are produced (e.g., by printing) and scored for allele counts and variants. Allele and mutant frequency are calculated using an SPPCR program. The model for this has been described in Coolbaugh-Murphy, et al. (2004). The mutant frequencies are compared between groups for significance using the arc-sin transformed mutant frequencies and the bootstrap standard error.

A SP-PCR examination of a sample consists of the amplification of one or more amounts of DNA; the result from each amount amplified is termed a “run.” Replicate samples of each amplification are conducted, each replicate is a “well.” The information obtained from a well consists of the identity of the alleles seen in it, for example, well 3 of run 1 might contain alleles 5 repeats and 20 repeats.

The operational unit of the amount of amplified DNA is the allele equivalent (a.e.): one a.e. is that amount of DNA that, when amplified, produces on average one identifiable allele. c denotes the number of a.e. in one experimenter DNA unit.

SP-PCR examination at a locus can be used to obtain information related to experimental design, such as the number of runs, the amount of DNA amplified in each run, and the number of wells; the identity of progenitor alleles; the number of wells in which each allele was seen for each run. Results of the statistical analysis will include, but is not limited to the calibration quantity, c (frequently, the amount of DNA that the experimenter amplifies at several loci is determined by the results at one locus, amplification may differ from locus to locus, so it is important to calibrate each separately); the frequency of each allele (the frequency of allele i is denoted by fᵢ.); the total mutation frequency and the variability of all estimates.

1. Overview of Statistical Methods.

The analysis of SP-PCR data uses maximum likelihood estimation as described in standard texts, for example Stuart and Ord, Kendall's Advanced Theory of Statistics, 1991. The steps in the development of methods for analyzing SP-PCR data include determining a statistical model. The model provides the probability of the outcome as a function of c and the fᵢ. This probability is termed the likelihood; its logarithm is the log-likelihood. An additional step includes choosing c and the fi to maximize the log-likelihood. A further step is computing the variance of the estimates.

2. Statistical Model

The number of alleles across all the wells fit a Poisson distribution, a standard model for the random number of particles in a fixed volume. The DNA amount is denoted (in experimenter units) in run r by Dᵣ. The mean number of alleles in each well of run r is cDᵣ. The probability that a random allele in a well is type i is fᵢ. Thus, the joint distribution of all allele types in a well is multinomial. Appendix A shows that with these assumptions, the distribution of the number of alleles of type in a well is Poisson with mean cDᵣ fᵢ. The probability of a particular number of type i alleles in a well is the same regardless of the number of alleles of a different type in the same well; restated, the numbers of different alleles in a well are independent.

Independence implies that the combination of numbers of types of alleles seen in wells provides no additional information about c or fi over the number of wells in which each allele type is seen. The probability of any combination is the product of the probabilities of each member of the combination. Thus the data relevant to fi is the number of wells in which allele i is seen and the number in which it is not seen, a considerable data reduction compared to all combinations of alleles per well. Independence of numbers of allele types in a well also implies that the mean number of alleles of different types in a well can be estimated separately for each allele type. This simplifies the computation.

a. Likelihood

If one lets μᵢ=cfᵢ. The mean number of alleles of type i in a well in-run r is Dᵣμᵢ The probability of not seeing allele i is thus
pᵤᵣ=exp(−Dᵣμᵢ), (1)

The probability of seeing allele i in a well is pₛᵣ=1−pᵤᵣ.

The probability of seeing allele i in nₛᵣ wells and of not seeing it in nᵤᵣ wells in a run is given by the binomial formula:

$${P}_{ir} = ( \frac{{n}_{sr} + {n}_{ur}}{{n}_{sr}} ) {p}_{sr}^{{n}_{sr}} {p}_{ur}^{{n}_{ur}} \qquad ( 2 )$$

This is the likelihood in allele i for run r.

Statisticians usually work with the logarithm of the likelihood instead of the likelihood itself, it is usually simpler and has theoretical advantages. The operations performed on log-likelihoods are maximization with respect to μᵢ and differentiation with respect to the parameters of the model. The logarithm of the binomial coefficient in the likelihood does not depend on any model parameters, only on the observed n's, so it is customarily omitted from the log-likelihood. The location of the maximum and the values of the derivatives with respect to model parameters are not changed by this omission.

The log-likelihood of seeing the i′th allele size in nₛᵣ wells and not seeing it in nᵤᵣ wells for run r is

$$\begin{matrix}{ll}_{ir} = {n}_{sr} log ( {p}_{sr} ) + {n}_{ur} log ( {p}_{ur} ) \\ = {n}_{sr} log ( 1 - exp ( - {D}_{r} {μ}_{i} ) ) - {n}_{ur} {D}_{r} {μ}_{i}\end{matrix} \qquad \begin{matrix}( 3 ) \\ ( 4 )\end{matrix}$$

where the last line follows by replacing pₛᵣ and pᵤᵣ from (1).

The total likelihood in i is the product of the probabilities, Pᵢᵣ, over all runs r. Logarithms transform products into sums, thus the log-likelihood for allele type i is

$${ll}_{i} = {\sum }_{r}^{} {ll}_{i} .$$

For any one run, r, the estimation of μᵢᵣ is straightforward. The maximum likelihood estimate of a binomial proportion of events is the observed proportion. Hence, the natural (and also maximum likelihood) estimate of μᵢ is obtained by solving

$${\hat{p}}_{ur} = \frac{{n}_{ur}}{{n}_{sr} + {n}_{ur}} = exp ( - {D}_{r} {\hat{μ}}_{i} ) \\ {\hat{μ}}_{ir} = - \frac{log ( {\hat{p}}_{ur} )}{{D}_{r}} \qquad ( 5 )$$

This yields \hat{μ}ᵢᵣ:

If there are several runs, the likelihood must be maximized numerically. A starting value for the maximization is the average over the runs of the \hat{μ}ᵢᵣ. The maximum likelihood estimate of μᵢ is denoted by \hat{μ}ᵢ.

If there is a limited number of runs, suppose that there is only one run in an assessment, of a sample and allele i was seen in every well. Then according to equation (5), the estimate of μᵢ is infinite. Theory provides no solution to this problem; any solution used will be ad hoc. The inventors solution is to increase nᵤᵣ from 0 to ½ and correspondingly decrease nₛᵣ. If there are several runs and nᵤᵣ is 0 in all of them, only the value in the run with the largest Dᵣ is modified.

3. Estimation of c. the fᵢ, and the Total Mutant Frequency

The estimate of c is

$$\hat{c} = {\sum }_{i}^{} {\hat{μ}}_{i} \qquad ( 6 )$$

since μᵢ=cfᵢ and Σᵢfᵢ=1.

The estimate of fᵢ is thus

$${\hat{f}}_{i} = \frac{{\hat{μ}}_{i}}{\hat{c}} \qquad ( 7 )$$

and the estimate of the fraction of mutants is:

$$\hat{m} = \frac{{\sum }_{k} {\hat{μ}}_{k}}{{\sum }_{j} {\hat{μ}}_{j}}$$

where j ranges over all alleles and k ranges over all mutant alleles.

4. Estimates of the Variances

There are two methods for computing the variance of the estimates, Asymptotic approximations or bootstrap estimates.

Asymptotic approximations. The accuracy of these approximations improves with increases in the total number of wells. This method has two disadvantages. 1) It requires a bit of mathematical sophistication to derive the estimates. 2) Theory does not provide methods for determining when the number of wells is sufficiently large for these approximations to be useful.

Simulation or bootstrap estimates. New random data is generated from the original data and it is fit to obtain estimates of c and the fᵢ. The process is repeated a large number (e.g., 1000) of times and the variance of the estimate is obtained from these replicate estimates.

In particular embodiments the simulation method is used because it does not require a large number of wells for accuracy; however, simulation requires more computation than the asymptotic method. With a modem computer the generation and analysis of 1000 random replicates of the experiment takes a fraction of a second. The generation of new random data sets proceeds as follows: For each run, the known number of wells and the probability of seeing an allele i in a well (pₛᵣ). The simulated value of nₛᵣ for allele i is a random binomial number in which the number of trials is the number of wells, and the probability of seeing allele i is pₛᵣ.

5. Transformation of Data

One of the primary uses for SP-PCR results is the comparison of mutation frequencies between specimens, for example, normal tissue versus tumor. The normal approximation to the binomial is frequently used to compare proportions. As the number of wells in SP-PCR gets larger, the normal approximation gets better. However, for any actual experiment, the approximation can be poor.

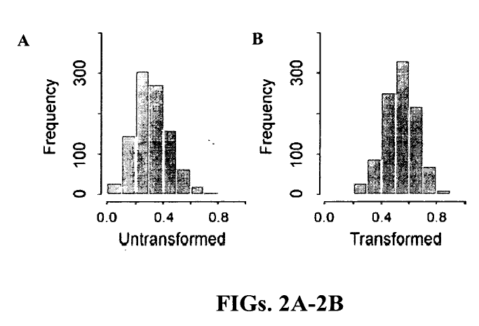

FIG. 2 (left panel) shows the distribution of 1000 random replicate estimates of a mutant frequency of 5%; the distribution is scaled from 0 to 1 to make it comparable to the rightmost panel. The distribution is notably skewed to the left; there are more values further from the mean on the left of the distribution than on the right. The right panel shows the distribution when the arcsin transform is applied to each estimate. The arcsin transformation of a proportion, m, is
t(m)=2arcsin((\sqrt{(m)})
and this transformation is frequently used to better approximate the normal distribution. The skew is less in the right panel than in the left; the left panel has a skewness of 0.61, the rightmost panel of −0.18. The skewness of a symmetric distribution would be zero, so the transformation slightly over corrects in this case.

6. Statistical Testing

Various statistical treatment of SP-PCR data can be undertaken. The most common statistical tests associated with SP-PCR include, but are not limited to:

b. Comparing Mutant Frequencies Between Two Specimens

Let the estimates of the transformed frequencies of interest in the two specimens be F₁ and F₂ and let the corresponding estimated variances be V₁ and be V₂. Then, since t(F₁) and t(F₂) are approximately normal, an appropriate statistic for assessing the significance of the difference between the two frequencies is:

$$Z = \frac{t ( {F}_{1} ) - t ( {F}_{2} )}{\sqrt{( {V}_{1} + {V}_{2} )}}$$

If the two frequencies are the same, then Z should be distributed as a unit normal, so a difference in absolute value of at least 1.96 is significant at the 0.05 level for a two-sided test.

c. Comparing Two Mutant Frequencies in a Single Specimen

The procedure includes the comparison of the μᵢ using a normal approximation. The μ's are independent.

d. Comparing Mutant Frequencies Between Two Categories of Specimens

Categories are groups of samples identifiable by some criterion; for example, samples from individuals with cancer and others without cancer or those with some genetic abnormality and those without the same abnormality. The transformation, t, that makes the data more nearly normal in one sample, is of no help here because the transformed mean of individual frequencies is not necessarily near the mean of the transformed frequencies. Another problem is that the variation estimated in one sample by the methods shown accounts only for the randomness inherent in SP-PCR. The specimen to specimen variation within a category adds to this variation. An appropriate transformation of the raw frequencies to make their distribution more nearly normal within categories is appropriate, but the transformation would vary from case to case. With or without such a transformation, the inventors would use the t-test and the signed-rank test and examine the data carefully if these two methods disagreed. Other statistical means of comparing two populations can be used to determine if the results of a particular study or assessment indicate the presence of a disease condition, the risk or increasing risk of a disease condition, or the initiation of a disease condition in a subject being tested.

D. Dilution for Identifying the Frequency of Mutant Fragments.

The methods take advantage of the reported (Zheng et al., 2000) statistical strength employing the Poisson distribution of alleles and likelihood models in calculating mutant frequencies in pools containing up to 100 haploid genomes. The inventors extended those methods to more accurately estimate the very low amounts of PCR amplifiable DNA allele by allele in sets of reactions ranging from <2->0.5 diploid g.e (see examples). This approach makes it unnecessary to do the extremely large numbers of pools studied by working at <0.5 g.e. for the accurate measurement of mutant frequencies (Leeflang et al., 1996).

E. Mutant Frequency as a Measure of MSI.

It is of note that the results are expressed as mutant frequencies, i.e., this is the calculated # of mutants observed of all the alleles examined. Results are not calculated as mutation frequencies and not mutation rates. While the latter processes will impact the frequency of mutant fragments, an immeasurable factor in the studies conducted on tumor material that will greatly impact mutant frequencies is the stage of tumor clonal evolution at which any particular mutant is fixed—the earlier in the stage a mutant becomes fixed, the greater the number of cells that carry that mutation (Tsao et al., 2000) and therefore the mutant frequency will be greater. But, by the methods presented, it is only mutant frequency that is assessed. In tumors having a high mutation rate there is a great probability of a mutation taking place at one of the microsatellite loci in the screen early in the clonal evolution of the tumor and therefore observable by the present methods. Thus, defining mutant frequency is a measure of MSI.

F. Verification

An argument can be made that when DNA is diluted down to 1 g.e. levels and nested PCR conducted to amplify the few target molecules present, contamination and PCR artifact can lead to identification of non-progenitor fragments present for reasons other than mutation. Aside from the precautions taken to limit contamination, the inventors present four approaches to validate non-progenitor fragments as mutants and not the result of artifact. 1) reconstruction experiments that show at greater dilutions of DNA result in better estimations of known “variant” fragments; 2) segregation experiments to verify recovery of known alleles at heterozygous loci at small pool levels; 3) split plate experiments in fragments are verified from a SP-PCR experiment on a replica plate made after the first few PCR reactions; and 4) the simultaneous use of normal controls with the application of the statistical approach presented in mutant frequency analysis of patient material.

Typically, after ABI GeneScan analysis, data are printed out as chromatograms to be scored for allele counts and variants. The data consists of whether or not each fragment was seen in every small pool. A model in which the number of alleles in replicate pools were distributed Poisson, and in which particular allele frequencies constituted a fixed proportion of the total has been described (Coolbaugh-Murphy et al., 2004). Maximum likelihood estimates of the mean number of alleles in each pool and the frequencies of each allele are derived. The mutant frequencies are compared between groups for significance using the arc-sin transformed mutant frequencies and the bootstrap standard error.

G. Computer implemented methods

A computer-implemented genomic analysis method for improving the robustness of SP-PCR and its derivative methods is provided in certain aspects of the invention. The computer-implemented analysis comprises the steps of receiving data input from a plurality of PCR amplification reactions and formatting that data for manipulation using a mathematical function, computing a variety of parameters including, but not limited to, a calibration quantity (c), allele frequency (f), total mutant frequency, as well as variances associated with such; computing the significance of alleles frequency within a sample and/or between a sample; computing the linkage of two or more markers, and the genetic instability of particular sample.

A computer-implemented genomic analysis method and system for genomic analysis on a single molecule scale is provided. The computer-implemented system comprises an data capturing device (e.g., fluorescent detector associated with capillary electrophoresis apparatus), and a computer having a memory and communicating with the data collection device, the computer capable of receiving and storing into the memory a plurality of electrophoresis results from the data capturing device, the computer being further capable of fitting a plurality of values associated with genomic amplifications to one or more mathematical function and computing an allele frequency and/or significance between allele frequencies.

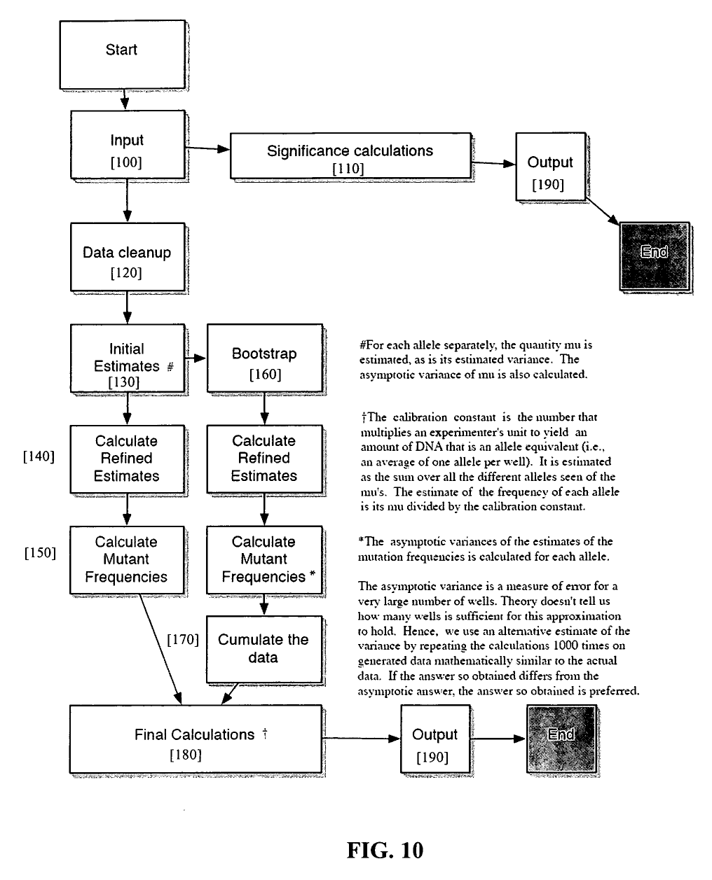

A flow chart illustrating a general program flow can be used in implementing the statistical method described herein (FIG. 10). Date will be formatted and presented to the computer as an input (100). The input will be processed by either performing a significance calculations (110) or by performing initial data clean algorithm (120). The data resulting from the data clean up (120) will be used to perform initial estimate calculations (130). Once initial estimates are determined the program can proceed by calculating refined estimates (140) followed by calculation of allele or mutant frequencies (150), which will be used in the final calculations (180). Alternatively or in parallel the initial estimates are used to perform the boot strap variance estimates (160) that can be followed by calculation of refined estimates (140) and allele or mutant frequencies (150). The alternative procedure will conclude with cumulating the data (170) and in the final calculations (180). The final calculation will include any verification, comparison, and/or significance calculations. The results will be formatted and prepared as an output (190).

II. Application of SP-PCR Methods

Genetic instability can be used as a marker for a variety of disease states or the risk of developing a disease state, such as cancer. In various embodiments of the invention the methods described herein are used as a surveillance tools in individuals who are at risk at developing a disease state, as a surveillance tool in patients that are at risk of developing resistance to certain drugs or therapies, and/or as a forensic or genomic analysis tools for constructing or reconstructing genetic progenitors or family trees.

A. Cancer Surveillance

The survival rate for cancer patients increases with early detection of cancer. Known methods of gaining early detection of cancer are limited to techniques such as surveillance endoscopy and random tissue biopsies, both of which are costly and inefficient. In addition, methods which employ relatively high levels of radiation which cause tissue damage generally are not preferred.

The development of cancer involves inactivation of many different types of genes in a cell. It is this inactivation that is largely responsible for a normal cell becoming a tumor cell. As a cell progresses to a hyperproliferative or cancerous state the genome becomes increasingly unstable. It is this instability in the genome that can be used as a biomarker for assessing risk of or progression to early stages of cancer. An increase in mutant DNA frequency is used a surveillance tool to indicate that more frequent or more thorough screening or assessment of a subject is needed. This type of surveillance would be useful in monitoring a members of family that are susceptible to certain cancers or have been in an environment that predisposes them to cancer or another hyperproliferative condition.

Assessment of MSI may also be used to monitor environmental genotoxic stress. For example, it is difficult to assess the long term effects of radiation exposure. However, by assessing MSI following radiation exposure the severity of genomic damage can be estimated and used to guide future diagnostic and preventative measures. Genomic damage caused by chemical agents in the environment can also be difficult to determine, especially in cases where the exposure level of an individual is unknown. MSI analysis may, in these cases, be used to indirectly determine an individuals level of exposure to environmental genotoxins. Furthermore, MSI analysis offers a more accurate method for determining the damage caused by genotoxin exposure. This information may be used to guide future health surveillance and direct preventative therapies.

Many cancer therapy strategies involve administration of genotoxic agents, such as DNA damaging chemicals and radiation. While such therapies are effective in combating cancers, systemic effects of the therapy can also result in MSI in non-cancerous tissue (Fonseca et al., 2005). Monitoring systemic MSI can therefore be used to estimate the amount of damage that chemotherapies cause to normal tissues. Thus, MSI analysis can be used to adjust chemotherapeutic regimens to reduce DNA damage in healthy tissues. Additionally, such analyses can be used to determine an individual's cancer risk later in life as a result of chemotherapy. Thus, the use of MSI in combination with cancer therapies may be used to minimize systemic genomic damage during the therapy and to better predict the future cancer risk of patients completing such therapies.

Since MSI analysis may be used to estimate the integrity of an individual's genome it can also be used to optimize clinical therapy for patients. For example, in cases where an individual exhibits a high levels of MSI cancer therapies may be selected that limit systemic genotoxic effects. Likewise, the use of drugs with known genotoxic effects may be limited in individuals that display high levels of MSI. MSI analysis may be used for example to better determine an individual's risk when administered estrogen therapy (i.e. to combat osteoporosis or the effects of menopause) (Liehr, 2000). Thus, in some cases MSI analysis can be used to determine individualized risk factors for administration of genotoxic agents to an individual.

B. Surveillance of Acquired Resistance to Therapy

A major obstacle to modem cancer therapies is acquired resistance to therapies. As discussed previously cancer cells can acquire high rates of mutation and therefore have an enhanced ability to acquire mutations that confirm resistance to anti-cancer therapies. Since MSI can be used to assess genome stability in cancer cells some aspects of the invention involve the use of MSI to estimate a mutation rate in a cancer and thus determine the probability that the cancer will acquire resistance to a therapy. Information gained from MSI analysis may be used to develop better therapeutic strategies for cancers with a high amount of genome instability. For example, such cancers may be simultaneously treated with multiple anti-cancer therapies to reduce the chance that the cancer will acquire resistance.

In yet further embodiments, MSI analysis may be used to identify cancers that have acquired resistance to a particular cancer therapy. For example, MSI data from particular loci may be used to determine whether a cancer cell is resistant to a therapy, such as a chemotherapy or an immunotherapy. In this instance, MSI analysis can be used to adjust the therapy administered to the individual, e.g. employing a different chemotherapeutic agent. In general it will be understood by one of skill in the art, that cancer cells with higher genome instability will be increasing resistance to DNA damaging therapies. Thus, in certain cases, MSI can be used to determine a cancer's resistance to therapies that induce DNA damage.

C. Phase Reconstruction

Alleles at two closely linked genetic loci travel together on the same chromosome. Therefore, the two maternal alleles at such loci would be on the same DNA fragment and the two paternal alleles would be on a different fragment. Two alleles on the same fragment are in “phase.” Even when the DNAs of parents are available, it is very difficult to determine phase of alleles at two different loci. Molecular haplotyping using the somatic cell DNA of individuals could provide such information. Making small pools (single genome equivalents), conducting total genome amplification of each pool, and performing PCR on the DNA of two closely linked loci, one can determine the phase of alleles at each locus—the two alleles that always appear in the same wells are in phase. Exceptions would be where the DNA fragment was broken (functions of the distance between the two loci and the quality of the DNA used for the analysis). A simple chi square test would determine if the difference from the expectation of phase was significant. Therefore SP-PCR facilitates molecular haplotyping.

Phase determination enables linkage disequilibrium mapping which is seen as a major approach to identifying the genetic factors involved in disease phenotypes (Botstein and Risch, 2003). Therefore much effort has been put into molecular haplotyping. Procedures have been developed (see review by Kwok and Xiao, 2004)—cloning, somatic cell hybrids, immobilizing DNA and others—but none can be said to be more simple and straight forward as the use of SP-PCR as described above.

D. Forensic Reconstruction

DNA analysis is now widely used as a highly accurate method of forensic analysis of evidence in criminal investigations. Since there are numerous aspects of individual DNA sequence that are unique a variety of forensic DNA analysis techniques have been developed and are currently in use.

Restriction Fragment Length Polymorphism (RFLP) is a technique for analyzing the variable lengths of DNA fragments that result from digesting a DNA a restriction endonuclease. The presence or absence of certain recognition sites in a DNA sample generates variable lengths of DNA fragments, which are separated using gel electrophoresis. Separated DNA is then hybridized with DNA probes that bind to a complementary DNA sequence in the sample. The patterns generated by this analysis are unique to the individual. However this technique has fallen out of favor since it require large amounts of intact DNA, often not available to forensic scientists.

Many modern forensic analyses involve polymerase chain reaction (PCR) analysis. PCR allows DNA analysis on biological samples as small as a few cells. With RFLP, DNA samples would have to be about the size of a quarter. The ability of PCR to amplify such tiny quantities of DNA enables even highly degraded samples to be analyzed. However, in the case of PCR great care must be taken to prevent contamination with other biological materials during the identifying, collecting, and preserving of a sample.

Short tandem repeat (STR) analysis is used to evaluate specific regions (loci) within nuclear DNA. Variability in STR regions can be used to distinguish one DNA profile from another. The Federal Bureau of Investigation (FBI) uses a standard set of 13 specific STR regions for CODIS. CODIS is a software program that operates local, state, and national databases of DNA profiles from convicted offenders, unsolved crime scene evidence, and missing persons. The odds that two individuals will have the same 13-loci DNA profile is about one in one billion.

Mitochondrial DNA analysis (mtDNA) can be used to examine the DNA from samples that cannot be analyzed by RFLP or STR. Nuclear DNA must be extracted from samples for use in RFLP, PCR, and STR; however, mtDNA analysis uses DNA extracted from another cellular organelle called a mitochondrion. While older biological samples that lack nucleated cellular material, such as hair, bones, and teeth, cannot be analyzed with STR and RFLP, they can be analyzed with mtDNA. In the investigation of cases that have gone unsolved for many years, mtDNA is extremely valuable.

MSI analysis as described herein may be used in combination with any of the forgoing techniques in forensic analysis procedures. However, MSI offers certain advantages relative to previously available techniques. First of all, methods described herein are able to resolve genetic information from a single strand of DNA and statistically analyze these results. Thus, MSI analyses can allow forensic scientist to determine whether a DNA sample is contaminated with DNA from more than one individual. Like many of the other forensic analysis techniques MSI may, in some cases, be used to determine genetically encoded attributes of an individual such as race, hair color, eye color or sex. However, MSI analysis also provides methods for determining information about a suspect that is not genetically encoded. For example forensic MSI analysis can be used to estimate the age an individual (Coolbaugh-Murphy, 2005).

Methods of forensic MSI analysis may be applied to any evidence that comprises samples with genetic material such as hair (root follicles), blood, tissue, bones, semen or teeth. For example, MSI may be used to genetically profile a body that can not be identified by other means. Additionally, MSI analysis can be used to estimate the age of an individual at the time of death. In certain other cases, material evidence at a crime scene by be analyzed and MSI used to genetically profile a suspect in the crime. In this case, the age of the suspect may be estimated thereby giving investigators additional physical information about a suspect. Thus, MSI techniques described herein may be used as a new forensic analysis tool that provides both genetic and physical information about the source genetic material.

E. Assessment of Microdeletions

In somatic cells small deletions are a form of genome instability that can lead to cancer. These can be detected by SP-PCR. DNA from a subject is quantified using a marker locus and diluted so that 1 genome equivalent (g.e.) of DNA (6 pg) is deposited into each well of 112 wells of a 384 well microtest plate. According to the Poisson distribution no fragments of the test locus is present in approximately 18 of the 112 wells. That is the usual result. However, if there was a deletion of one of the alleles, the number of wells not containing such a fragment would be doubled and easily statistically identified. This can be done without need of the locus in question having two different alleles (heterozygous). Therefore, one can observe “loss of heterozygosity” without the locus under study being heterozygous.

The inventors have identified such an event in cancer cells of Li-Fraumeni patients where a microsatellite locus on chromosome 17 did not produce the expected number of fragments after SP-PCR. Analysis of that region of the chromosome indicated that the locus, D17S250, was surrounded by Alu repeat sequences. Such regions are prone to deletion. SP-PCR will allow further study of such phenomena.

### EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Estimating Allele Frequencies by SP-PCR

I. Materials and Methods

Patient DNA. Two MSI-High individuals were studied. Patient B a 40 y/o male and part of a kindred meeting the most stringent criteria for HNPCC (Boland et al., 1998). This patient had a colorectal cancer (CRC) and negative immunostaining for hMSH2—uncommon in sporadic cancers. He was diagnosed with CRC at the young age of 42 and his mother was diagnosed with cancer of the biliary tract at the age of 40. Biliary cancer is considered a part of the HNPCC syndrome. Both the patient and his mother have Muir-Torre syndrome which is characterized by cutaneous lesions seen in a subset of HNPCC patients. While a specific MSH2 mutation has not yet been found in this kindred, the syndrome as described identifies the patient as a carrier of a germ line mutation for CRC predisposition. Patient C was a 74 y/o sporadic colon cancer patient with no family history of the disease but whose MLH1 gene was methylated in the promoter of the tumor DNA (Frazier et al., 2003).

Normal control DNA. These were selected from 426 normal control PBLs obtained from the University of Texas M.D. Anderson Blood Bank identified by gender (212 females and 214 males) and age (range 18 to 67 y/o, with at least 8 samples for each year of life). They were genotyped for the loci used in the SP-PCR analysis.

SP-PCR high-throughput methodology. A “run” for each tumor included constitutive tissue from outside the tumor site and PBLs from an age, gender, and allele size matched control. Each tissue in each run was studied for six microsatellite locus and for each locus there were 96-112 small pools at approximately 1.0 g.e./pool. This definition of a “run” applies in all experiments unless otherwise noted. PBLs from the patients were routinely used as a constitutive tissue control, while normal distal colon was available only from Patient B.

The flow of procedures in a “run” is summarized as follows. Upon receipt of the DNAs, genotyping and initial quantification was conducted. Genotyping of the patient constitutive material, and age-matched candidate normal controls was done by standard PCR at the multiple loci. The multiplexed PCR products were analyzed on the ABI 3100 (ABI, Foster City, Calif.). The patient samples and candidate normal controls were quantified at the beta-globin locus using the Roche LightCycler and the LightCycler Control Kit (Roche, Indianapolis, Ind.). This was done to determine the amount of amplifiable DNA present in the samples. The DNAs were then diluted to single diploid genome levels.

Multiple hemi-nested SP-PCRs were conducted on sets of three DNAs—the patient constitutive and tumor, and matched normal control. Approximately >100 alleles distributed over 96-112 PCR replicates per sample were amplified at each locus, with the three DNAs on one 384-well tray per locus. Negative controls of water-PCR mix occupied the remaining wells on the tray. The use of the MWG Biotech RoboSeq 4204 S robot with an onboard Primus-HT 384 thermocycler (MWG Biotech, High Point, N.C.) was used for the setup and amplification of the initial “outer” PCR and the distribution of the secondary “inner” PCR. To minimize the possibility of contamination, initial PCR 384-well trays were transferred from a “low-copy” PCR area to a “high-copy” PCR area, where they were diluted using the Qiagen BioRapidPlate and Twister I robots (Valencia, Calif.). This allowed the initial PCR dilution and transfer of the diluted product to the secondary PCR's 384-well tray in a matter of minutes, something not possible for the number of wells, trays, and loci examined if done manually. The secondary “inner” PCR was also amplified using another MWG Biotech Dualblock Primus-HT 384 thermocycler (High Point, N.C.). Four μl of the products from representative wells for each sample at each locus were examined on a 2% agarose minigel for estimation of product yield to determine volumes for multiplex analysis. The RapidPlate was again used to multiplex all the loci's 384-well trays for each sample set, resulting in 384 wells, each containing the SP-PCR products of multiple loci. Thus, 2 μl to 10 μl (depending on the estimated yield) of well number 1 from each tray (for each locus) was combined into well number 1 of the multiplex tray, and so on for all 384 wells. The use of robotics at this point not only was timesaving, it was essential to prevent pipetting errors and contamination. Robots were hooded and under positive pressure with UV light decontamination. The trays of multiplexed SP-PCR products were submitted to the UT-MDACC DNA Core facility, where they were analyzed on an ABI 3100 capillary system, running GeneScan software. The ABI system takes advantage of the use of multiple fluorescent dyes used to end-label one of the inner-PCR primers. Loci products with overlapping sizes were labeled with different fluorescent dyes, allowing them to be multiplexed and co-electrophoresed. The dyes used on the loci for this project were 6-FAM, NED, and VIC on the primers, and ROX for the internal size standard on the ABI.

After ABI Genescan analysis, the data were printed out as chromatograms, to be scored for allele counts and variants. The allele counts were used to determine the Poisson distribution of the DNAs examined, and using the program “SPPCR-Calibrate,” the number of diploid g.e. examined in that run was estimated at each locus. The quantification at the D2S123 locus was used as a reference for each DNA. Then, using the g.e. data and the number of variants observed, the program estimated the mutant frequency for each tissue in that run at each locus. The mutant frequencies were compared between the normal control and the patient samples for significance using the arc-sin transformed mutant frequencies and the bootstrap standard error.

Listed below are exemplary primer sequences for the loci used—Forward, “F”, Reverse, “R”, Inner, “I”, Outer, “O”)

BAT 25:

(SEQ ID NO:1)

FO-5′-tca tgg agg atg acg agt tg;

(SEQ ID NO:2)

RI/O-5′-tgg ctc taa aat gct ctg ttc tc;

(SEQ ID NO:3)

FI-5′Vic-5′-tcg cct cca aga atg taa gtg;
BAT 26:

(SEQ ID NO:4)

FO-5′-gtt tga act gac tac ttt tga;

(SEQ ID NO:5)

RI/O-5′-cca atc aac att ttt aac c;

(SEQ ID NO:6)

FI.5′Fam-5′-tga cta ctt ttg act tca gcc;
D2S123:

(SEQ ID NO:7)

FO-5′-tga cca aaa gca ttt ctc tta tg;

(SEQ ID NO:8)

RI/O-5′-cct ttc tga ctt gga tac cat cta tct;

(SEQ ID NO:9)

FI.5′Fam-5′-aaa cag gat gcc tgc ctt ta;:
D55346:

(SEQ ID NO:10)

FO-5′-tga gaa atg aaa tc gaat gga g;

(SEQ ID NO:11)

RI/O-5′-tca ggg aat tga gag tta cag gt;

(SEQ ID NO:12)

FI-5′Ned-5′- ggc ctg gtt gtt tcc cta gta t;
D17S250:

(SEQ ID NO:13)

RO-5′-aag gct gag gca act gat gt;

(SEQ ID NO:14)

FI/O-5′-cac ata cat aaa ctt tca aat ggt ttc a;

(SEQ ID NO:15)

RI-5′Vic-5′-tcc gaa agt gct ggg att ac;
DMPK:

(SEQ ID NO:16)

C.FO-5′-tct ccg ccc agc tcc agt c;

(SEQ ID NO:17)

ER.RO-5′-cag gcc tgc agt ttg ccc atc;

(SEQ ID NO:18)

H.FI-5′FAM-5′-aac ggg gct cga agg gtc ctt;

(SEQ ID NO:19)

DR.RI-5′-aaa tgg tct gtg atc ccc cca.

Amplification reactions for all loci except DMPK (Monckton et al., 1995; Monckton et al., 1997) used 1X GeneAmp/Gold buffer (ABI, Foster city, Calif.), 1.5-3.5 mM MgCl₂, (SIGMA, St. Louis, Mo.,) 250 μM dNTPs (Amersham Pharmacia, Piscataway, N.J.,) 1 μM each forward and reverse primers, 1U AmpliTaq Gold polymerase, (ABI, Foster city, Calif.), and ˜0.5-2.0 g.e. DNA in a final volume of 10 μl per reaction. Cycle times were 95° C.×6 min., [(95° C.×45 sec, 55° C.×45 sec, 70° C.×1 min 45 sec)×30 cycles], 70° C.×7-10 min, hold 4° C.∞. Initial amplification products were diluted 10-fold, with 2 μl used as template in the secondary amplification reactions.

II. Results

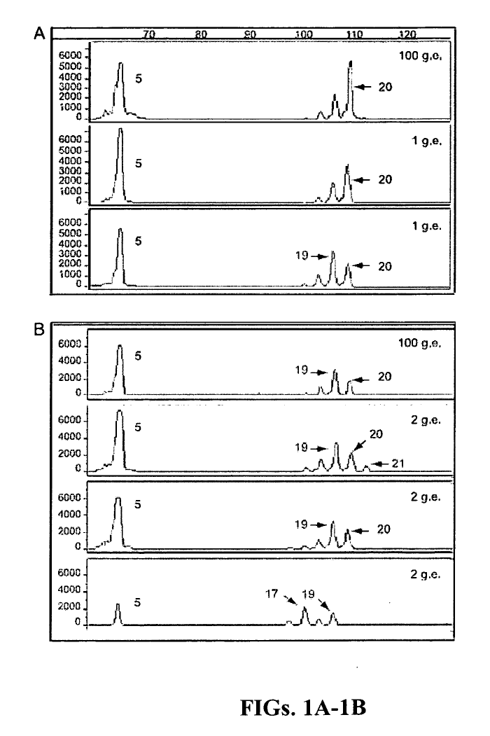

Identification of mutant fragments by SP-PCR. FIG. 1A shows chromatogram data of the trinucleotide repeat in the DMPK locus amplified from a section of “normal” colon from an HNPCC MSI-H patient and demonstrates how mutant fragments may be present in a sample and seen by SP-PCR yet not visible in traditional PCR. This is especially true for one repeat deletions (common events in this material) since the new mutant fragment falls into the stutter fragment of its progenitor allele. The mutant fragment, being infrequent in this sample, cannot be seen in the top panel which has over 100 g.e. In FIG. 1B, similar data using the colon tumor of the patient indicates why traditional PCR flagged this sample as a possible MSI-H—the mutant 19-repeat fragment was in such abundance as to be visible by traditional PCR (100 g.e., top panel). The small pool data will allow the counting of mutant fragments and frequency calculations in addition to identifying mutant fragments less frequent than the most frequent one. Statistical considerations for doing that are presented herein.

Mutant frequency calculations. The PBLs of a normal control blood donor was examined at the DMPK locus to determine whether there is a non-zero frequency of mutant alleles. The data is shown in the first three rows of Table 1. Three types of alleles were seen, two progenitor and one mutant. DNA amounts are shown in investigator estimated units which were the initial estimates of g.e.

TABLE 1
Example data and analysis.

| DNA^{a} | N wells^{b} | Progenitor^{c} | Progenitor^{d} | Mutant^{e} |
|---|---|---|---|---|
| 2.0 | 28 | 23 | 23 | 1 |
| 1.6 | 48 | 42 | 41 | 4 |
| 0.6 | 96 | 10 | 2 | 0 |
| μ initial estimate | | 0.782 | 0.70 | 0.024 |
| μ max likelihood | | 0.623 | 0.526 | 0.027 |
| estimate | | | | |
| Allele frequency | | 0.530 | 0.448 | 0.022 |
| Asymptotic SE of | | 0.043 | 0.043 | 0.010 |
| frequency | | | | |
| Bootstrap SE of | | 0.030 | 0.030 | 0.010 |
| frequency | | | | |

^{a}DNA per well in investigator units
^{b}number of wells in this run
^{c}number of wells in which progenitor allele 1 was seen
^{d}number of wells in which progenitor allele 2 was seen
^{e}number of wells in which the mutant allele was seen

One can readily calculate the initial estimate of the μ's. For example, for progenitor allele 1, the three runs in order give, according to formula 5

$$\hat{μ} = - \frac{log ( 5 / 28 )}{2} = 0.861$$

$$\hat{μ} = - \frac{log ( 6 / 48 )}{1.6} = 1.300$$

$$\hat{μ} = - \frac{log ( 86 / 96 )}{0.6} = 0.183$$

The mean of these three values is the initial estimate of the μ of Progenitor 1 allele (fourth row of Table 1). The maximum likelihood estimate is obtained by varying this value to maximize the log-likelihood for each allele. Computer methods provide the maximum likelihood estimate of 0.623 for Progenitor allele 1 (fifth row of Table 1).

From formula 6,
ĉ=0.623+0.526+0.027=1.176

The amount of PCR amplifiable DNA in a run is thus estimated to be about 18% more than the investigator's initial estimate.

Estimates of the allele frequencies are readily obtained using formula 7—the results are shown in the sixth row of the Table 1. The final two rows of the Table 1 show the asymptotic and bootstrap estimates of the standard errors of the allele frequencies.

Using the bootstrap standard errors, we can compute a 95% confidence interval on the frequency of the mutant allele: 0.022+1.96*0.01=(0.0024, 0.0416). From the bootstrapped transformed values (not shown), the interval is (0.0067, 0.0482).

Comparison with other methods. Three methods of analysis have appeared in the literature.

Method 0: (Zheng et al., 2000)—The model is similar to the present model; however, the authors consider only the case of a single run (DNA amount) and two-alleles and use only asymptotic methods to obtain variances. The inventors use a more extensive analysis including an arbitrary number of alleles while demonstrating that the analysis can be performed allele by allele rather than all at once which greatly simplifies both the requisite data capture and the analysis. Also the inventors include the bootstrap method of estimating variances as an alternative to and check on the asymptotic methods.

Apparently simpler methods of analysis are possible if one assumes that when a particular allele is seen in a well, there is only one such allele in the well. Unfortunately, this assumption is incorrect.

Method 1: (Yao et al., 1999)—The number of wells in which an allele is seen is divided by the total number of alleles examined to estimate the frequency of the allele.

If the estimate of c, determined using the present methods, in the example is used to calculate the total number of alleles examined; this number is c×Dᵣ×number of wells=1.485×2×20=59.4. Allele 5 was seen in 16 wells, so the estimate of f₅ is
f₅=16/59.4=0.269

Similarly, f₁₉=0.051, and f₂₀=0.236.

Method 2: (Bacon et al., 2001)—This method is described in Bacon et al. as “. . . the frequency of mutant alleles in each sample was expressed as the number of alleles that were mutant in length divided by the total number of alleles detected (normal and mutant). Accordingly the frequency of mutants was not the exact number of cells with alterations but represents the relative proportions of alleles.”

The total number of alleles detected in the example was the sum of the number of wells in which each allele was seen, i.e., 16+14+3=33. From this, f₅=16/33=0.484, f₁₉=0.091, and f₂₀=0.422.

The inventors compared these methods with the present methods using 1000 random data sets generated using parameters fit by the present methods to the example. Thus, the correct answers are known. Each of the 1000 data sets was fit by the three methods; the results are shown in Table 2. The mean results of the present methods do not precisely replicate numbers used to generate the data, although they come quite close. These results are the average over 1000 data sets, probably none of which is identical to that used to obtain the parameters.

TABLE 2
Comparison of three methods

| | | Mean frequency estimate | | |
|---|---|---|---|---|
| Allele^{a} | Frequency^{b} | Method 0 | Method 1 | Method 2 |
| 5 | 0.540 | 0.544 | 0.270 | 0.486 |
| 19 | 0.055 | 0.055 | 0.051 | 0.091 |
| 20 | 0.404 | 0.401 | 0.235 | 0.423 |

^{a}Allele size.
^{b}Frequency of allele in generated data.

The example used to compare methods is the small pool data of FIG. 1. Twenty wells were run at an investigator estimated DNA amount of 2 a.e. Of these, 16 contained the 5 repeat fragment, 14 contained the 20 repeat fragment, and 3 contained the mutant 19 repeat fragment. The estimates of the parameters obtained by the methods of the previous sections are: c=1.485, f₅=0.540, f₁₉=0.055, and f₂₀=0.404.

Method 1 provides a good estimate of the frequency of mutants. It does a poor job of estimating the frequencies of the progenitor alleles since they cluster in fewer wells than there are alleles. As a consequence of this clustering, the frequencies do not add to 1.

Method 2 is an improvement—the frequencies add to 1. However, it gives an estimate of mutation frequency that is twice the correct value. The problem is that the denominator in the frequency calculation is too small—there are more alleles total than the sum of the number of wells in which each allele is seen.

Table 3 quantifies the clustering of alleles in wells. It shows the probability of more than one allele in a well and the mean number of alleles in a well given that at least one allele was present in the well. At very low a.e. the mean number of alleles in a well in which one or more is detected is not much greater than one; at larger a.e., the number is much greater than one.

TABLE 3
Probability of more than one allele and mean number
of alleles in a well given at least one allele

| μ^{a} | Prob 2^{b} | Mean No.^{c} |
|---|---|---|
| 0.5 | 0.229 | 1.271 |
| 1.0 | 0.418 | 1.581 |
| 1.5 | 0.569 | 1.930 |
| 2 | 0.686 | 2.313 |
| 3 | 0.842 | 3.157 |
| 4 | 0.925 | 4.075 |
| 5 | 0.966 | 5.034 |

^{a}Mean number of alleles per well.
^{b}Probability of two or more alleles in a well given that there is at least one allele in the well.
^{c}Mean number of alleles in a well given that there is at least one allele in the well.

The values in Table 3 explain the difficulties in Methods 1 and 2. Note that according to the estimate of c, the data was obtained at about 3 a.e. The progenitor arrays have a frequency near ½, which gives a mean number of each progenitor allele per well of 1.5. According to the third row of Table 3, there are nearly two progenitor alleles in each well in which one is seen. The use of this figure would bring the Progenitor frequency estimate of Method 1 near to ½. There are only, on average, 0.138 alleles of size 19 per well, so there are, on average, 1.07 alleles in each well in which one or more is seen. Consequently, the estimate of the frequency of allele 19 by Method 1 is fairly accurate. A similar argument applies to Method 2.

Rather than attempt modifications of Methods 1 or 2, the inventors recommend the use of the present method based on maximum likelihood. Maximum likelihood is the universally preferred form of estimation of statisticians. An early version of a computer program for the various calculations described is available as F95 source and Win32 and Macintosh OS9 executables. sppcr in our alphabetical list of available software at odin.mdacc.tmc.edu/anonftp/.

Effective level of dilution for identifying the frequency of mutant fragments. As a first example of the application of the statistical approach, here is presented an analysis of the most appropriate g.e. levels for identifying mutant fragments by SP-PCR. SP-PCR was conducted on varying amounts (g.e.) of DNA from “normal” constitutive tissue and tumor tissue from an HNPCC MSI high patient in order to determine the level of DNA most effective for detecting mutant fragments. As seen in Table 4, as the input genome equivalents (g.e.) decreased from 100 to ˜1.0 g.e., the ability to detect and quantify mutants increased inversely. Going to lower than 1.0 g.e. required doing greater numbers of small pools without increasing the efficiency (data not shown). Alleles observed on the chromatograms were counted and logged into allele distribution datasheets, with separate rows for each experiment and each DNA input amount. The expected g.e., number of reactions, and numbers of each progenitor and variants were entered into the SP-PCR statistical analysis program (above). The program's results included the estimated g.e., the mutant frequency (MF), and the transformed standard error (SE). The estimated g.e. was used to calculate the number of amplifiable alleles screened in a given dataset (e.g.: n as reported in Table 5). The SE is used to determine the significance of the difference between the patient sample MF and the normal control MF.

TABLE 4
Number of pools with different size DMPK alleles following SP-PCR at decreasing g.e.
from “normal” constitutive colon (top) and colon tumor (bottom) from an HNPCC patient
and frequencies (MF) of mutant fragments detected

| | | Est No. of | | | | | | | | | | |
|---|---|---|---|---|---|---|---|---|---|---|---|---|
| | No. | alleles | No. of pools showing the following alleles^{a} | | | | | | | | | |
| Est g.e. | pools | screened | 66 | 90 | 93 | 102 | 105 | 108 | 111 | 114 | 117 | MF^{b} |
| Patient B normal colon | | | | | | | | | | | | |
| 10.0 | 4 | 800 | 4 | | | | | 4 | 4 | | | 0 |
| 50 | 3 | 300 | 3 | | | | | | 3 | | | 0 |
| 20 | 3 | 160 | 3 | | | | | 4 | 4 | | | 0 |
| 10 | 10 | 200 | 9 | | | | | | 9 | 1 | | 0.005 |
| 0.8-1.0 | 94 | 157 | 65 | | | | | 3 | 51 | 1 | | 0.026* |
| Patient B colon tumor | | | | | | | | | | | | |
| 100 | 3 | 600 | 3 | | | | | 3 | 3 | | | 1.00 |
| 50 | 3 | 300 | 3 | | | | | 3 | 3 | | | 1.00 |
| 20 | 4 | 160 | 4 | | | | | 4 | 4 | | | 1.00 |
| 10 | 10 | 200 | 10 | | | | | 7 | 10 | 1 | 1 | 0.109* |
| 1.0 | 142 | 289 | 125 | 1 | 1 | 2 | 3 | 80 | 75 | 2 | | 0.485* |

^{a}Given as number of base pairs of the observed fragments instead of numbers of repeats. The numbers of base pairs for progenitor alleles (5 repeats and 20 repeats) of this patient are shown in bold (66 and 111 bp, respectively).
^{b}Significant (p < 0.01) MF are shown in italic with an asterisk. Significance was determined from simultaneous analysis of two normal control PBL DNAs, 218 pools at f1.37 estimated ge, 462 estimated alleles, 5 variants, MF 0.011.

TABLE 5
Summary of SP-PCR data for patients B and C and matched normal controls at 6 loci

| | Loci | | | | | | | | | | | |
|---|---|---|---|---|---|---|---|---|---|---|---|---|
| | DM1 | | D2S123 | | D5S346 | | D17S250 | | BAT25 | | BAT26 | |
| Tissue | n | f | n | f | n | f | n | f | n | f | n | f |
| Patient B | | | | | | | | | | | | |
| Control PBLs | 462 | <0.01 | 267 | <0.01 | 381 | <0.01 | 219 | 0.04 | 273 | 0.01 | 137 | <0.01 |
| Patient PBLs | 148 | 0.03 | 241 | 0.08* | 179 | 0.06* | 164 | 0.11* | 100 | 0.04 | 104 | 0.02 |
| Patient colon | 201 | 0.03* | 133 | 0.09* | 194 | 0.01 | 135 | 0.09 | 138 | 0.04 | 278 | <0.01 |
| Colon CA | 972 | 0.26* | 169 | 0.31* | 176 | 0.53* | 136 | 0.59* | 183 | 0.57* | 278 | <0.01 |
| Patient C | | | | | | | | | | | | |
| Control PBLs | 86 | 0.09 | 128 | 0.06 | 179 | 0.04 | 150 | 0.04 | 153 | 0.03 | 126 | <0.01 |
| Patient PBLs | 165 | 0.08 | 130 | 0.04 | 123 | 0.00 | 144 | 0.04 | 137 | <0.01 | 118 | <0.01 |
| Colon CA | 150 | 0.35* | 114 | 0.25* | 147 | 0.53* | 130 | 0.46* | 120 | 0.65 | 141 | <0.01 |

Estimated number of alleles (n) and mutant frequency (f) are shown.
*Mutant frequencies significantly different from normal controls are in italics.

The procedure takes advantage of the reported (Zheng et al., 2000) statistical strength employing the Poisson distribution of alleles and likelihood models in calculating mutant frequencies in pools containing up to 100 haploid genomes. Here, the present methods have been expanded to include to more accurately estimate the very low amounts of PCR amplifiable DNA allele by allele in sets of reactions ranging from <2->0.5 diploid g.e. This approach makes it unnecessary to do the extremely large numbers of pools studied by working at <0.5 g.e. for the accurate measurement of mutant frequencies (Leeflang et al., 1996).

Mutant frequency as a measure of MSI. Note that the results are expressed as mutantfrequencies, i.e. this is the calculated # of mutants observed of all the alleles examined. Results are not calculated as mutation frequencies and not mutation rates. While the latter processes will impact the frequency of mutant fragments, an immeasurable factor in the studies conducted on tumor material that will greatly impact mutant frequencies is the stage of tumor clonal evolution at which any particular mutant is fixed—the earlier in the stage a mutant becomes fixed, the greater the number of cells that carry that mutation (Tsao et al., 2000) and therefore the mutant frequency will be greater. But, by the methods presented, it is only mutant frequency that can be measure. The best measure would be mutation rate—the rate at which mutations are produced during some standard interval, usually per cell division. However, in tumors having a high mutation rate there is a great probability of a mutation taking place at one of the microsatellite loci in the screen early in the clonal evolution of the tumor and therefore observable by the present methods. Thus, defining mutant frequency is a measure of MSI.

Reconstruction Studies. A mutant frequency of 0.06 was reconstructed by mixing DNAs from two different individuals having two different DMPK genotypes—B, a 5 repeat/20 repeat heterozygote considered the “progenitor” genotype for this reconstruction; and A, a 10 repeat/12 repeat heterozygote considered the “mutant” genotype. The mixture contained 94% genotype B and 6% genotype A. SP-PCR was conducted at 50 g.e., 20 g.e., 5 g.e., 2 g.e. and 0.8 g.e. Alleles of genotype A were recovered at levels approaching the expected 0.06 only at the 2 g.e. and 0.8 g.e. levels (frequencies of 0.058 and 0.08 respectively). At higher DNA concentrations the frequency of recovery of genotype A alleles never exceeded 0.026. Therefore, the 1 to 2 g.e level was effective in identifying infrequent fragments at their appropriate frequency.

Segregation analysis. For SP-PCR to be effective in mutation analysis, all the alleles in a sample must be represented amongst the small pools. This was tested by studying the 4 polymorphic loci in the screen on a series of control PBLs heterozygous for the loci—(D2S123, 6 individuals; D5S346, 4 individuals; D17S250, 6 individuals; and DMPK, 7 individuals). At each locus and for each individual there was a larger allele and a smaller allele, Single-cell PCR studies in the field of preimplantation genetics have demonstrated the phenomenon of “allele drop out” where the larger of the two alleles does not amplify at a locus being subjected to single cell PCR (Rechitsky et al., 2001). This could be due to the larger allele not amplifying as robustly (artifact), or to a deletion, rearrangement, mutation, or other such perturbation. In studies of tumor DNA using SP-PCR such an observation could have similar meaning, hence the need to establish that normal allele segregation is observed at the loci used in this study. Larger vs. smaller allele recovery over the loci ((D2S123, 308 vs. 282; D5S346, 186 vs. 179; D17S250, 229 vs. 245; and DMPK, 578 vs. 516) showed no significant differences in the distribution of alleles at heterozygous loci into wells or in the amplification of the different sized alleles. While there appears to be no overall significant distortion of recovery of alleles it should be noted that the greatest difference in recovery of smaller rather than larger alleles of the heterozygote appeared at the DMPK locus. Most of that difference can be attributed to the cases where there was a large discrepancy between the sizes of the two alleles (5 repeat and 20 repeat, data not shown)—a factor that must be considered in such experiments.

Split Plate analysis of SP-PCR detected mutants and artifacts. To determine that the SP-PCR assay itself was not introducing artifact or “false positive” fragments, the procedures of those who tested that concept in the evaluation of single-molecule nested fluorescent PCR of microsatellite repeats in sperm DNA (Zhang et al., 1994) were used. The usual SP-PCR procedure was followed using the DMPK microsatellite locus except that after 3 cycles the thermocycler was paused and the parent plate (A) was placed on ice and one-half the reaction volume for each well was transferred to a “replicate” plate, (B), which was sealed, and stored at 4C. The parent plate A was then sealed and SP-PCR analysis completed. Empty wells, wells with variants and wells with progenitor alleles were identified on the parent plate A. The replicate plate B was then put through the rest of the SP-PCR procedure and well analysis to determine if variants, progenitors, or empty wells from plate A produced the same results on plate B. Therefore in each experiment one was able to determine the MF as well as the artifact frequency. Experiments were conducted with four normal control PBL DNAs (from patients around 40 y/o and the one tumor DNA sample from the MSI-high HNPCC patient of the same general age. MFs for the four controls were 0.01, 0.01, 0.02 and 0.02 and the artifact frequencies in each case were <0.01, 0.03, 0.02 and 0.02 respectively. For the tumor sample the MF was 0.23 and the artifact frequency was 0.01. Therefore, in any typical study in which 165 alleles (110 replicates at 0.75 g.e.) were screened it would be possible to distinguish mutant frequencies greater than background by 0.03 as significant (p<0.01).

Statistical approach for determination of mutant frequencies. As a test of the molecular and statistical procedures in evaluating MFs, the tumor and constitutive tissue of two MSI colon cancer patients—one an HNPCC (by definition carrying a germ line mutation predisposing to colon cancer) and one a sporadic in which there was methylation of the MLH1 gene in the tumor tissue (Frazier et al., 2003)—were studied. Simultaneously run (on the same microtest plates) were age, gender and allele size matched normal control DNAs from PBLs of blood bank volunteers. Controls were matched for those parameters since there is evidence that MSI increases with age (Wong et al., 1995; Fortune et al., 2000), fragment size of progenitor allele (Zhang et al., 1994; Sia et al., 1997) and gender (Zhang et al., 1994; Boyd, 1996). These were studied at six different microsatellite loci—five from the loci recommended in such analyses (Boland et al., 1998), and a locus typically used as a standard—DMPK. A concern was the use of PBLs from normal individuals as controls for SP-PCR artifact since it could be suggested that instability might be introduced into such DNA in the fixation procedure. This is a concern that does not have any experimental basis. However, to allay any such concern, several studies have been done to determine whether fixation increases MSI in the DNA. For one, sectioned fixed tonsil material was used and run through the SP-PCR procedure for all loci in the screen and found no difference in MSI between that material and PBL controls (data not shown). Secondly, as seen in Table 5, the fixed normal colon of Patient B does not show any higher MSI than the fresh frozen PBLs of the same patient. In all these studies, the fixed material DNA was found to contain a lower fraction of amplifiable DNA than DNA from the fresh frozen samples. However, that is dealt with in the dilutions necessary to make the small pools.

From Table 5, it is clear why the tumor material from both patients was identified as MSI-H—in 5 of the 6 loci tested the frequency of mutant fragments was equal to or >0.26. Such levels are detectable by standard PCR. Meaningful MSI (<0.25-0.05) exist in the tumors of the at least 50% of non-polyposis colon cancer patients meeting all the criteria of inherited disease yet not having MSI detectable by traditional PCR (Weisner et al., 2003). SP-PCR on a greater patient base should provide an opportunity to explore that issue.

Seen also is the first identification of MSI in the constitutive tissue of the patient with the germ line mutation since that made by Parsons et al. (1994). Here statistically significant MSI are seen at four different loci in two different tissues—PBLs and “normal” colon. The significance of the observation is underscored by the fact that the phenomenon was not observed in the individual with the sporadic cancer and therefore not having a germ line mutation for colon cancer predisposition. One could point to the fact that the mutant frequencies in the PBLs of patient C were a bit elevated. However, MSI does increase with age (Wong et al., 1995; Fortune et al., 2000) and so when the frequencies of MSI in the PBLs of this elderly person (74 y/o) are compared with the frequencies in the age and gender matched control PBLs, they are not significantly different. One explanation of MSI in patient B's PBLs is that the MSI is due to escaped and circulating tumor cells. One would expect a similar observation in the sporadic patient C. That is not the case therefore that explanation is not supported by the data. A more likely explanation is that the constitutive cells of the person bearing a germ line mutation for a cancer causing disease might have some aspect of the phenotype. This exciting possibility, and its consequences of perhaps identifying persons at risk in inherited situations where a mutation in a specific gene has not been identified will have to await further studies.

### Example 2

### MSI in PBL DNA of MSI-High HNPCC Patients Carrying Germline MMR Gene Mutations.

Microsatellite instability (MSI) has been well documented in tumor DNA from hereditary non-polyposis colon cancer (HNPCC) patients known to carry germline mutations in major mismatch repair genes. It has been hypothesized that such germline mutations might also result in lower, yet detectable clinically significant levels of MSI in constitutive tissues, (Parsons et al., 1995). To that end, the inventors used small-pool PCR to examine PBL and tumor DNA from seven microsatellite instability-high (MSI-H) HNPCC patients—3 with MLH1 and 4 with MSH2 mutations, age 36-71 yr. (Table 6). Alteration types included splice, missense, deletions, and stop mutations. Each patient was studied at D2S123, D5S346, and D17S518, previously shown to be informative for quantitative MSI analysis, (Coolbaugh-Murphy et al., 2005).

TABLE 6

| ID | Age at sampling | Gene | Mutation Type |
|---|---|---|---|
| 1 | 41 | hMLH1 | Missense |
| 2 | 48 | hMLH1 | Splice |
| 3 | 71 | hMLH1 | Nonsense/stop |
| 4 | 36 | hMSH2 | Splice |
| 5 | 46 | hMSH2 | Deletion |
| 6 | 59 | hMSH2 | Missense |
| 7 | 42 | hMSH2 | IHC negative |

All seven patients demonstrated increased constitutive MSI in at least 2 of the 3 loci examined by SP-PCR. Two of the three mutation carrying patients in both the MLH1 and MSH2 mutant groups showed significantly increased PBL MSI at all 3 loci. The cumulative weighted 3 locus average mutant frequency (MF) for the 3 MLH1 patients was 0.11 in PBL and 0.19 in tumor, ranging from 0.04 to 0.23 in PBL and 0.12 to 0.31 in tumor. For the 3 MSH2 patients, the cumulative 3 locus average MF was 0.12 and 0.26 for PBL and tumor DNA, respectively, ranging from 0.05 to 0.20 in PBL and 0.07 to 0.43 in tumor. Normal Control's PBL cumulative 3 locus average MF was 0.01, and ranged from 0.00 to 0.04. From the 7 patients, no clear trends yet emerge regarding different MF patterns or levels resulting from different genes affected, different types of mutations, or patient age. For the MLH1 patients, the D2S123 and D5S346 loci were informative in the PBL DNA of all 3 patients, D17S518 was significant in the PBL DNA of 2 patients. For the MSH2 patients, again, D5S346 was significant in the PBL of all 3 patients, while D2S123 and D17S518 were significant in 2 of the 3 patients. The data suggest that regardless of the MMR gene affected or the type of mutation leading to MSI-H in tumors, SP-PCR can be used as a functional assay to measure low level genomic instability of clinical significance in constitutive tissue.

Prior study of one older, sporadic CRC patient, with an epigenetic MLH1 promoter methylation, showed that the MF's in that patient's PBL DNA were not significantly different from that in age-matched normal control PBL DNA—even when 6 loci were scrutinized, (Coolbaugh-Murphy et al., 2004). Subsequent analysis of 7 more older, sporadic CRC patients' DNA at 3 loci showed MF's ranging from 0.00-0.06, with a 3-locus average ranging from 0.00-0.04, not significantly different from previous and concurrently analyzed age-matched normal control PBL DNAs. Overall, these observations support the conclusion that individuals carrying germline mutations predisposing them to HNPCC exhibit significantly increased levels of MSI in their constitutive tissues, which can be tested, measured, and monitored.

I. Materials and Methods

Patient DNA. We used small-pool PCR to examine PBL and tumor DNA from seven microsatellite instability-high (MSI-H) HNPCC patients—3 with inherited MLH1 and 4 with MSH2 mutations, age 36-71 yr. Alteration types included splice, missense, deletions, and stop mutations. Each patient was studied at D2S123, D5S346, and D17S518, previously shown to be informative for quantitative MSI analysis, (Coolbaugh-Murphy et al., 2005). The inventors also analyzed of 8 older, (age 65-80 years old) sporadic CRC patients' PBL DNA.

Normal control DNA. These were selected from 426 normal control PBLs obtained from the University of Texas M.D. Anderson Blood Bank identified by gender (212 females and 214 males) and age (range 18 to 67 y/o, with at least 8 samples for each year of life). They were age and gender matched to the patients used in the SP-PCR analysis.

SP-PCR high-throughput methodology. The general methodology was as described herein, particularly as described in Example 1. Briefly, a “run” for each HNPCC patient included DNA from tumor and constitutive tissue (PBL) and PBLs from an age, gender, and allele size matched control. Each tissue in each run was studied for three microsatellite loci and for each locus there were 96-112 small pools at approximately 0.75 g.e./pool. This definition of a “run” applies in all experiments unless otherwise noted. Additional primers include D17S518FO.2 5′-tctttatagcattagtctctgggaca (SEQ ID NO:20); D17S518FI.2.5′FAM 5′-tagtctctgggacacccaga (SEQ ID NO:21); D17S518.RI/O 5′-gatccagtggagactcagag (SEQ ID NO:22).

II. Results

The inventors contemplate that mutations in DNA MMR genes result in the measurable increases of MSI in the DNA of “normal” somatic cells. To identify a molecular phenotype in normal cells that identify people at increased risk for cancer small pool PCR (SP-PCR) (FIG. 3 and FIG. 4) was performed to quantify MSI in PBL DNA by diluting DNA to single genome equivalents and conduct microsatellite PCR on over 100 such small pools so that mutant microsatellite fragments as infrequent as 1% can be identified and counted. PBL DNA of 7 HNPCC patients with known germline mutations, their age matched unrelated normal controls, and PBL DNA of 8 patients with sporadic CRC (who do not have predisposing germline mutations). Tumor DNA of HNPCC patients was also included as positive controls.

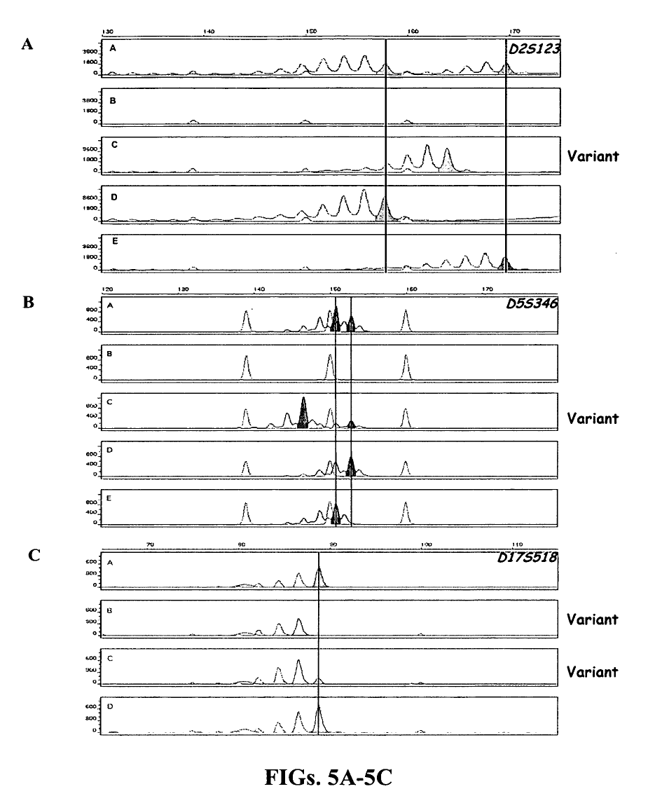

Representative chromatograms of small pools of the 3 microsatellite loci used in the analysis are illustrated in FIG. 5. Samples used were heterozygous for D2S123 and D5S346 and homozygous for D17S518. Vertical lines show positions of progenitor alleles. In some pools both heterozygous progenitor alleles were captured (panel A of D2S123 and D5S346) In some pools no alleles were present (panel B of D2S123 and D5S346); Individual progenitor alleles were segregated (panels D and E of D2S123 and D5S346); Mutant alleles were captured either alone (panels C of D2S123, B of D17S518) or with a progenitor allele (panel C of D5S346 and D17S518).

Significant levels of MSI in PBL DNA from germline mutation carriers was seen. SP-PCR data from MSI-High germline mutation carrying HNPCC patients and unrelated age-matched normal controls is presented in Table. 7. Mutant Frequencies observed in DNA from patient tumor and peripheral blood DNA were compared the control's peripheral blood DNA. Loci examined were D2S123, D5S346, and D17S518. Number of estimated alleles observed are indicated by (n), while (m) is the number of variants seen, and (f) is the mutant frequency. The last column contains the weighted (f) average for each tissue at all 3 loci. Mutant frequencies in patient tissues which are significantly different from controls are identified by a p<0.01. As seen in the Table 7, blood DNA from the all 7 mutation carriers showed significant mutant frequency levels (0.04 to 0.15, avg=0.11) of MSI by this approach, and this was not observed using traditional PCR. Normal controls showed very low background mutant frequency levels (0.00-0.04) consistent with not carrying inherited mutations which predispose one to cancer. Tumor DNA from these same patients, used as a positive control, demonstrated 3 loci average mutant frequency scores of 0.07-0.43, with an overall average of 0.25, as expected for an MSI-H tissue.

TABLE 7
MSI in Blood DNA of MSI-H HNPCC patients with germline mutations

| | | | | | | | | | | | 3 loci weighted | | |
|---|---|---|---|---|---|---|---|---|---|---|---|---|---|
| | | D2S123 | | | D5S346 | | | D17S518 | | | average | | |
| ID | Tissues | n | m | f* | n | m | f* | n | m | f* | n | m | f* |
| 1 | Control PBLs | 251 | 4 | 0.02 | 262 | 2 | 0.01 | 257 | 1 | 0.00 | 770 | 7 | 0.01 |
| | Patient PBLs | 277 | 14 | 0.05 | 333 | 17 | 0.05 | 214 | 3 | 0.01 | 824 | 34 | 0.04 |
| | Tumor | 220 | 98 | 0.65 | 167 | 6 | 0.04 | 340 | 3 | 0.01 | 727 | 107 | 0.15 |
| 2 | Control PBLs | 392 | 5 | 0.01 | 580 | 0 | 0.00 | 409 | 1 | 0.00 | 1381 | 6 | 0.00 |
| | Patient PBLs | 204 | 11 | 0.05 | 222 | 41 | 0.21 | 168 | 4 | 0.02 | 594 | 56 | 0.09 |
| | Tumor | 86 | 8 | 0.09 | 132 | 28 | 0.21 | 106 | 4 | 0.04 | 324 | 40 | 0.12 |
| 3 | Control PBLs | 270 | 0 | 0.00 | 384 | 0 | 0.00 | 582 | 0 | 0.00 | 1236 | 0 | 0.00 |
| | Patient PBLs | 127 | 46 | 0.42 | 79 | 11 | 0.14 | 307 | 59 | 0.23 | 513 | 116 | 0.23 |
| | Tumor | 73 | 26 | 0.36 | 117 | 45 | 0.38 | 305 | 80 | 0.26 | 495 | 151 | 0.31 |
| 4 | Control PBLs | 172 | 3 | 0.02 | 141 | 10 | 0.07 | 178 | 5 | 0.03 | 491 | 18 | 0.04 |
| | Patient PBLs | 121 | 12 | 0.10 | 51 | 24 | 0.47 | 130 | 23 | 0.18 | 302 | 59 | 0.20 |
| | Tumor | 100 | 32 | 0.32 | 30 | 22 | 0.73 | 115 | 35 | 0.31 | 245 | 89 | 0.36 |
| 5 | Control PBLs | 142 | 3 | 0.02 | 65 | 1 | 0.02 | 173 | 2 | 0.01 | 380 | 6 | 0.02 |
| | Patient PBLs | 151 | 10 | 0.07 | 99 | 8 | 0.08 | 191 | 3 | 0.02 | 441 | 21 | 0.05 |
| | Tumor | 145 | 8 | 0.06 | 97 | 9 | 0.09 | 119 | 8 | 0.07 | 361 | 25 | 0.07 |
| 6 | Control PBLs | 83 | 3 | 0.04 | 90 | 2 | 0.02 | 128 | 8 | 0.06 | 301 | 13 | 0.04 |
| | Patient PBLs | 155 | 1 | 0.01 | 94 | 17 | 0.18 | 126 | 37 | 0.29 | 375 | 55 | 0.15 |
| | Tumor | 40 | 13 | 0.33 | 60 | 44 | 0.73 | 132 | 43 | 0.33 | 232 | 100 | 0.43 |
| 7 | Control PBLs | 267 | 1 | <0.01 | 381 | 5 | 0.01 | 219 | 9 | 0.04 | 867 | 15 | 0.02 |
| | Patient PBLs | 241 | 18 | 0.07 | 179 | 11 | 0.06 | 164 | 17 | 0.10 | 584 | 46 | 0.08 |
| | Tumor | 169 | 39 | 0.23 | 176 | 93 | 0.53 | 136 | 65 | 0.48 | 481 | 197 | 0.41 |
| Sum | Control PBLs | 1577 | 19 | 0.01 | 1903 | 20 | 0.01 | 1946 | 26 | 0.01 | 5426 | 65 | 0.01 |
| | Patient PBLs | 1276 | 112 | 0.09 | 1057 | 129 | 0.12 | 1300 | 146 | 0.11 | 3633 | 387 | 0.11 |
| | Tumor | 833 | 224 | 0.27 | 779 | 247 | 0.32 | 1253 | 238 | 0.19 | 2865 | 709 | 0.25 |

MSI in PBL DNA of sporadic CRC patients without germline mutations compared to MSI in PBL DNA of HNPCC patients and normal control PBL DNA is illustrated in Table 9. SP-PCR data is shown from sporadic, non-germline mutation carrying CRC patients, unrelated age-matched normal controls, and MSI-H positive controls from previous Table 7. Mutant frequencies observed in DNA from patient peripheral blood DNA were compared to the normal and positive control's peripheral blood DNA. Loci examined were D2S123, D5S346, and D17S518. The number of estimated alleles observed are indicated by (n), while (m) is the number of variants seen, and (f) is the mutant frequency. The last column contains the weighted (f) average for each tissue at all 3 loci. Mutant frequencies in patient tissues which are significantly different from controls are indicated by a p<0.01. Note that none of the sporadic CRC samples show significant MSI, while the HNPCC patient PBL DNA demonstrated consistency, again showing significant Levels of MSI by SP-PCR analysis.

TABLE 8
Summary data on sporadic CRC patients.

| Blood | | | | | | | | | | |
|---|---|---|---|---|---|---|---|---|---|---|
| Sample # | Gender | Age | Polymorphism | Gene | Exon | Cdn | Nucleotide | Heterozygous | Homozygous | Notes |
| spor1 | Male | 65 | Y | hMSH2 | 10 | | A to T | Y | N | |
| spor6 | Male | 68 | | | | | | | | No mutations |
| | | | | | | | | | | identified. |
| spor3 | Female | 75 | Y | hMSH2 | 6 | 322 | GGC to | | | Not |
| | | | | | | | GAG | | | pathological |
| spor4 | Female | 80 | Y | hMSH2 | 10 | | T to A | Y | N | 9 bases |
| | | | | | | | | | | before exon |
| | | | | | | | | | | 10 |
| spor7 | Male | 76 | Y | hMSH2 | 10 | | A to T | N | Y | A/A |
| spor5 | Male | 72 | | | | | | | | No mutations |
| | | | | | | | | | | identified. |
| spor2 | Male | 70 | Y | hMSH2 | 10 | | T to A | N | Y | A/A |
| spor8 | Male | 73 | N | hMLH1 | | | | | | promoter |
| | | | | | | | | | | methylation |

TABLE 9
MSI in PBL DNA of Sporadic CRC patients without germline mutations compared to
MSI in PBL DNA of HNPCC patients and normal control PBL DNA.

| | ID | D2S123 | | | D5S346 | | | D17S518 | | | 3 loci weighted avg. | | |
|---|---|---|---|---|---|---|---|---|---|---|---|---|---|
| | Tissues | n | m | f** | n | m | f** | n | m | f** | n | m | f** |
| nc264 | NC PBLs | 88 | 5 | 0.06 | 79 | 1 | 0.01 | 95 | 0 | 0.00 | 6 | 262 | 0.023 |
| TA029 | Spor 1 PBLs | 383 | 5 | 0.01 | 210 | 1 | 0.00 | 250 | 3 | 0.01 | 9 | 834 | 0.011 |
| TA789 | Spor 2 PBLs | 486 | 0 | 0.00 | 330 | 0 | 0.00 | 947 | 2 | 0.00 | 2 | 1763 | 0.001 |
| 2 or 3* | Positive PBLs | 31 | 16 | 0.51 | 38 | 6 | 0.16 | 176 | 32 | 0.18 | 54 | 245 | 0.220 |
| nc380 | NC PBLs | 132 | 5 | 0.04 | 257 | 6 | 0.02 | 177 | 2 | 0.01 | 13 | 566 | 0.023 |
| TA176 | Spor 3 PBLs | 83 | 2 | 0.02 | 108 | 6 | 0.06 | 95 | 3 | 0.03 | 11 | 286 | 0.0389 |
| TA390 | Spor 4 PBLs | 375 | 3 | 0.01 | 375 | 2 | 0.01 | 217 | 2 | 0.01 | 7 | 967 | 0.007 |
| 2 or 3* | Positive PBLs | 56 | 2 | 0.04 | 53 | 10 | 0.21 | 104 | 9 | 0.09 | 21 | 213 | 0.099 |
| nc343 | NC PBLs | 124 | 3 | 0.02 | 88 | 0 | 0.00 | 111 | 0 | 0.00 | 3 | 323 | 0.009 |
| TA722 | Spor 5 PBLs | 486 | 2 | 0.00 | 205 | 0 | 0.00 | 300 | 0 | 0.00 | 2 | 991 | 0.002 |
| TA151 | Spor 6 PBLs | 514 | 1 | 0.00 | 237 | 1 | 0.00 | 340 | 0 | 0.00 | 2 | 1091 | 0.002 |
| 2 or 3* | Positive PBLs | 65 | 14 | 0.22 | 35 | 6 | 0.17 | 240 | 14 | 0.06 | 34 | 340 | 0.100 |
| nc343 | Control PBLs | 22 | 1 | 0.04 | 51 | 5 | 0.10 | 28 | 0 | 0.00 | 6 | 101 | 0.059 |
| TA666 | Spor 7 PBLs | 176 | 0 | 0.00 | 3219 | 1 | 0.00 | 184 | 2 | 0.01 | 3 | 681 | 0.004 |
| 2 or 3* | Positive PBLs | 21 | 4 | 0.19 | 35 | 10 | 0.29 | 46 | 7 | 0.15 | 21 | 102 | 0.206 |
| nc406-7 | NC PBLs | 128 | 7 | 0.06 | 179 | 7 | 0.04 | 150 | 6 | 0.04 | 20 | 457 | 0.044 |
| Pt. C | Spor 8 PBLs | 130 | 5 | 0.04 | 123 | 0 | 0.00 | 144 | 6 | 0.04 | 11 | 397 | 0.028 |
| | All Control | 494 | 21 | 0.0 | 654 | 19 | 0.03 | 561 | 8 | 0.01 | 48 | 1709 | 0.028 |
| | PBLs | | | | | | | | | | | | |
| | Sporadic PBLs | 2633 | 18 | 0.0 | 1909 | 11 | 0.01 | 2477 | 18 | 0.01 | 47 | 7019 | 0.007 |
| | Positive PBLs | 173 | 36 | 0.2 | 161 | 32 | 0.20 | 566 | 62 | 0.11 | 130 | 900 | 0.144 |

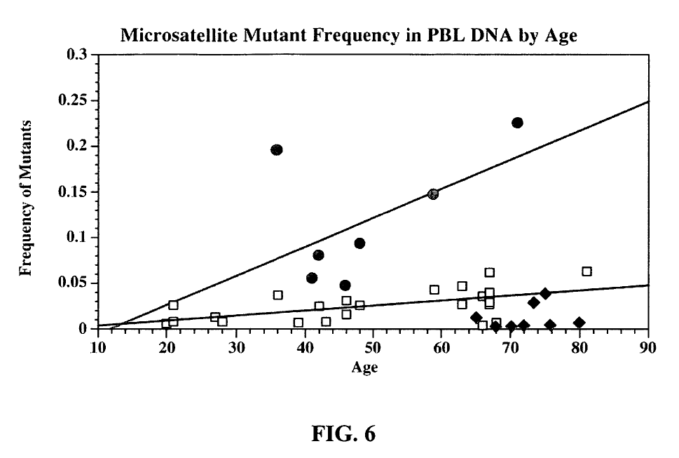

A graphic representation of the detectable and quantifiable differences in blood DNA mutant frequencies is shown in FIG. 6. This demonstrates the range of normal MF's as one ages, and the increase in MF when one carries predisposing mutation(s), i.e., those seen in HNPCC. Because the sporadic cases do not carry such predisposing mutations, their PBL DNA does not show an increase in MF over that associated with increasing age.

Overall, the data from Tables 7 and 9, and FIG. 6 demonstrate the sensitivity and the specificity of the assay for detecting early, systemic, low-level genomic DNA mutations that, in this example, are the downstream effect of decreased DNA repair capacity as a result of a mutation in a gene or genes that are responsible for maintaining genomic integrity. Because the tissue examined was non-tumor, the data indicate that pre-tumor analysis of those that carry mutations is feasible. Because the DNA source was white blood cells from peripheral blood, this suggests that other non-invasive sources of white blood cells, such as saliva, would also be suitable for such analysis. Traditional PCR has not been able to detect, describe, nor quantify early, rare, cumulative genomic DNA changes such as those seen by the SP-PCR approach to MSI analysis.

MSI levels in blood DNA of HNPCC patients are statistically significantly higher than the levels seen in age matched controls or in the PBL DNA of sporadic CRC patients. These observations support the hypothesis that individuals carrying germline mutations in DNA repair genes predisposing them to cancer also exhibit significantly increased levels of MSI in the DNA of their constitutive tissues, which can be tested, quantified, and monitored.

### Example 3

### MSI Increases with Age in Normal Somatic Cells

I. Materials and Methods

Subject DNA. Subjects were selected from 426 normal control PBLs obtained from the University of Texas M.D. Anderson Blood Bank identified by gender (212 females and 214 males) and age (range 18-67 y/o, with at least 8 samples for each year of life). Seventeen were randomly selected from this study and they fell into three age categories, (6 were 20-30 y/o, 5 were 35-50 y/o and 6 were 60-70 y/o). They are listed under “Subject” in Table 10.

TABLE 10
Frequencies of MSI at six microsatellite loci in the PBLs of normal individuals in three different age categories.

| | | | | Loci | | | | | | | | | | | |
|---|---|---|---|---|---|---|---|---|---|---|---|---|---|---|---|
| Subject | | | | No. of estimated alleles (n), no. mutants (m), mutant frequency (f)^{a} | | | | | | | | | | | |
| Age | | | | BAT26 | | | D2S123 | | | D5S346 | | | DMPK | | |
| Group | Individual | Age | Gender | n | m | f | n | m | f | n | m | f | n | m | f |
| 20-30 | 349 | 20 | f | 263 | 0 | <0.001 | 386 | 0 | <0.001 | 145 | 4 | 0.028 | 630 | 2 | 0.003 |
| | 350 | 20 | m | 612 | 0 | <0.001 | 843 | 9 | 0.012 | 613 | 4 | 0.007 | 1036 | 1 | <0.001 |
| | 388 | 21 | f | 326 | 0 | <0.001 | 267 | 5 | 0.020 | 173 | 13 | 0.094 | 444 | 2 | 0.004 |
| | 342 | 21 | m | 423 | 0 | <0.001 | 428 | 6 | 0.019 | 167 | 1 | 0.006 | 481 | 6 | 0.015 |
| | 28 | 27 | m | 133 | 0 | <0.001 | 503 | 1 | 0.002 | 282 | 8 | 0.033 | 360 | 9 | 0.024 |
| | 13 | 28 | f | 127 | 0 | <0.001 | 512 | 4 | 0.010 | 410 | 2 | 0.008 | 301 | 2 | 0.007 |
| | | Total^{b} | n = 6 | 1884 | 0 | 0.000 | 2939 | 25 | 0.009 | 1790 | 32 | 0.018 | 3252 | 22 | 0.007 |
| 35-50 | 105 | 39 | m | 87 | 0 | <0.001 | 239 | 4 | 0.017 | 167 | 2 | 0.019 | 110 | 9 | 0.165 |
| | 20 | 42 | f | 137 | 0 | <0.001 | 326 | 4 | 0.012 | 223 | 3 | 0.013 | 336 | 10 | 0.030 |
| | 148 | 43 | m | 169 | 0 | <0.001 | 273 | 4 | 0.019 | 288 | 1 | 0.006 | 341 | 4 | 0.015 |
| | 40 | 46 | m | 102 | 0 | <0.001 | 157 | 11 | 0.074 | 144 | 2 | 0.016 | 147 | 4 | 0.027 |
| | 9 | 48 | m | 163 | 0 | <0.001 | 112 | 5 | 0.046 | 210 | 7 | 0.04 | 343 | 5 | 0.026 |
| | | Total^{b} | n = 5 | 658 | 0 | 0.000 | 1107 | 28 | 0.025 | 1032 | 15 | 0.015 | 1277 | 32 | 0.025 |
| 60-70 | 318 | 63 | m | 122 | 2 | 0.017 | 193 | 7 | 0.026 | 114 | 8 | 0.072 | 173 | 4 | 0.023 |
| | 421 | 63 | f | 152 | 1 | 0.007 | 190 | 8 | 0.025 | 117 | 5 | 0.045 | 413 | 13 | 0.031 |
| | 264 | 66 | m | 112 | 0 | <0.001 | 184 | 6 | 0.033 | 200 | 10 | 0.050 | 122 | 4 | 0.033 |
| | 406 | 67 | m | 81 | 0 | <0.001 | 201 | 4 | 0.023 | 163 | 3 | 0.026 | 142 | 5 | 0.036 |
| | 407 | 67 | m | 126 | 0 | <0.001 | 166 | 8 | 0.048 | 125 | 5 | 0.040 | 124 | 10 | 0.086 |
| | 340 | 67 | m | 161 | 0 | <0.001 | 351 | 8 | 0.025 | 115 | 9 | 0.094 | 236 | 1 | 0.004 |
| | | Total^{b} | n = 6 | 754 | 3 | 0.004 | 1285 | 41 | 0.032 | 834 | 40 | 0.048 | 1210 | 37 | 0.031 |

| | | | | Loci | | | | | | Totals^{b} | | | | | |
|---|---|---|---|---|---|---|---|---|---|---|---|---|---|---|---|
| | | | | No. of estimated alleles (n), | | | | | | Mean 6 loci | | | Mean 3 loci^{c} | | |
| Subject | | | | no. mutants (m), mutant frequency (f)^{a} | | | | | | | | 6 loci | | | 3 loci |
| Age | | | | D17S250 | | | D17S518 | | | Total | Total | weighted | Total | Total | weighted |
| Group | Individual | Age | Gender | n | m | f | n | m | f | n | m | avg. | n | m | avg. |
| 20-30 | 349 | 20 | f | 332 | 1 | 0.002 | 261 | 1 | 0.003 | 2017 | 8 | 0.004 | 792 | 5 | 0.006 |
| | 350 | 20 | m | 725 | 11 | 0.021 | 669 | 0 | <0.001 | 4498 | 25 | 0.006 | 2125 | 13 | 0.006 |
| | 388 | 21 | f | 222 | 8 | 0.041 | 251 | 0 | <0.001 | 1683 | 28 | 0.017 | 691 | 18 | 0.026 |
| | 342 | 21 | m | 437 | 12 | 0.033 | 299 | 0 | <0.001 | 2235 | 25 | 0.011 | 894 | 7 | 0.008 |
| | 28 | 27 | m | 385 | 7 | 0.009 | 121 | 3 | 0.025 | 1784 | 28 | 0.016 | 906 | 12 | 0.013 |
| | 13 | 28 | f | 142 | 4 | 0.027 | 164 | 3 | 0.019 | 1656 | 15 | 0.009 | 1086 | 9 | 0.008 |
| | | Total^{b} | n = 6 | 2243 | 43 | 0.019 | 1765 | 7 | 0.004 | 13873 | 129 | 0.009 | 6494 | 64 | 0.010 |
| 35-50 | 105 | 39 | m | 189 | 4 | 0.023 | 528 | 1 | 0.002 | 1320 | 20 | 0.015 | 934 | 7 | 0.007 |
| | 20 | 42 | f | 121 | 9 | 0.084 | 205 | 12 | 0.061 | 1348 | 38 | 0.028 | 754 | 19 | 0.025 |
| | 148 | 43 | m | 99 | 6 | 0.062 | 164 | 1 | 0.006 | 1334 | 16 | 0.012 | 725 | 6 | 0.008 |
| | 40 | 46 | m | 449 | 8 | 0.019 | 144 | 1 | 0.007 | 1143 | 26 | 0.023 | 445 | 14 | 0.031 |
| | 9 | 48 | m | 400 | 4 | 0.013 | 210 | 2 | 0.010 | 1438 | 23 | 0.016 | 532 | 14 | 0.026 |
| | | Total^{b} | n = 5 | 1258 | 31 | 0.025 | 1251 | 17 | 0.014 | 6583 | 123 | 0.019 | 3390 | 60 | 0.018 |
| 60-70 | 318 | 63 | m | 118 | 9 | 0.083 | 184 | 8 | 0.042 | 904 | 38 | 0.042 | 491 | 23 | 0.047 |
| | 421 | 63 | f | 198 | 15 | 0.078 | 242 | 2 | 0.008 | 1312 | 44 | 0.034 | 549 | 15 | 0.027 |
| | 264 | 66 | m | 161 | 11 | 0.071 | 206 | 5 | 0.024 | 985 | 36 | 0.037 | 590 | 21 | 0.036 |
| | 406 | 67 | m | 132 | 5 | 0.038 | 121 | 6 | 0.051 | 840 | 23 | 0.027 | 485 | 13 | 0.027 |
| | 407 | 67 | m | 336 | 12 | 0.036 | 114 | 12 | 0.107 | 991 | 47 | 0.047 | 405 | 25 | 0.062 |
| | 340 | 67 | m | 306 | 4 | 0.013 | 106 | 6 | 0.060 | 1275 | 28 | 0.022 | 572 | 23 | 0.040 |
| | | Total^{b} | n = 6 | 1251 | 56 | 0.045 | 973 | 39 | 0.040 | 6307 | 216 | 0.034 | 3092 | 120 | 0.039 |
| ^{a}The mutant frequency is not simply product the number of observed mutants × 1/number of estimated alleles. The SP-PCR program, takes into account the Poisson distribution of alleles, and the likelihood that there may be multiple copies of a given allele in any given well. In addition, the program utilizes a bootstrap analysis of the mutant frequency, resulting in the generation of a number of random data sets similar to the | | | | | | | | | | | | | | | |
| # real data set. Combined, this results in the estimated total number of alleles and a bootstrap mutant frequency with a smaller standard error, thus a better estimate of the mutant frequency of a given tissue at a given locus. | | | | | | | | | | | | | | | |

^{b}Totals are of the sums of the estimated alleles and mutants at each locus and for each age group. Therefore, mutant frequencies of the totals are the products of the number of observed mutants × 1/number of estimated alleles.
^{c}Loci are D2S123, D5S346 and D17S518.

SP-PCR methodology. These methods are describe in detail above. Essential aspects are summarized here. Upon receipt of the DNAs, genotyping was done by standard PCR for the six microsatellite loci in the study, the mononucleotide repeat, BAT26; dinucleotide repeats, D2S123, D5S346, D17S250, D17S518; and the trinucleotide repeat, DMPK. The multiplexed PCR products were genotyped on the ABI 3100 (ABI, Foster City, Calif.). Samples were quantified at the beta-globin locus using the Roche LightCycler™ and the Light-Cycler Control Kit™ (Roche, Indianapolis, Ind.) in order to determine the amount of amplifiable DNA present in the samples. The DNAs were then diluted to approximately single diploid genome levels.

Multiple hemi-nested SP-PCRs were conducted on sets of three DNAs, one from each age group. Approximately 100 alleles distributed over 96-112 PCR replicates per sample were amplified at each locus, with the three DNAs on one 384-well tray per locus. The use of the MWG Biotech RoboSeq 4204 S™ robot with an onboard Primus-HT 384™ thermocycler (MWG Biotech, High Point, N.C.) was used for the setup and amplification of the initial “outer” PCR and the distribution of the secondary “inner” PCR after which they were diluted using the Qiagen BioRapidPlate™ and Twister I™ robots (Valencia, Calif.). The secondary “inner” PCR was also amplified using another MWG Biotech Dualblock Primus-HT 384™ thermocycler (High Point, N.C.). The RapidPlate™ was again used to multiplex all the loci's 384-well trays for each sample set, resulting in 384 wells, each containing the SP-PCR products of multiple loci. The trays of multiplexed SP-PCR products were submitted to the UT-MDACC DNA Core facility, where they were analyzed on an ABI ₃₁₀₀™ capillary system, running GeneScan™ software. The dyes used on the loci for this project were 6-FAM, NED and VIC on the primers, and ROX for the internal size standard on the ABI. The primers and dyes used for each locus were described in Coolbaugh-Murphy et al. (2004) except for D17S518. For that locus they were FO-50-tctttatagcattagtctctgggaca (SEQ ID NO:20); RI/O-50-gatccagtggagactcagag (SEQ ID NO:21); FI-6FAM-50-tagtctctgggacacccaga (SEQ ID NO:22) where F, forward; R, reverse; O, outside and I, inside.

Data analysis. After ABI GeneScan™ analysis, the data were printed out as chromatograms to be scored for allele counts and variants. The data consists of whether or not each fragment was seen in every small pool. A model in which the number of alleles in replicate pools were distributed Poisson, and in which particular allele frequencies constituted a fixed proportion of the total has been described (Coolbaugh-Murphy et al., 2004). Maximum likelihood estimates of the mean number of alleles in each pool and the frequencies of each allele were derived. The mutant frequencies were compared between groups for significance using the arc-sin transformed mutant frequencies and the bootstrap standard error.

A logit transformation was necessary for a linear regression analysis in order to plot age against mutant frequencies. That is because the best linear regression fit for age and frequency (f) produced a model in which negative values of f had a large probability for young ages, less than 18 years of age. No data for normals in this age group were available and there may be an age before which no mutations occur. Therefore, a model positing that mutant frequency, f, was a linear function of age (f=a+b×age) was not acceptable since the best fit for a and b produced a model in which negative values of f had a large probability for young ages. The transformation, y=1n (f/(1−f)) eliminated this problem since it maps values of f from 0 to 1 onto y values of −infinity to infinity. Since a number of observed mutant frequencies were zero, a small number (arbitrarily chosen as 0.001) was added to f in this transformation. A model in which this y was posited to be linear in age (y=a+b×age) appeared to be reasonable and to be an acceptable representation between mutant frequency and age. In particular, a non-parametric smooth of the data was obtained. The inventors used a method called “loess” (Cleveland and Devlin, 1988) to perform this smoothing. This method makes no assumptions about the form of the representation of mutant frequency with age, and the results suggested no form that would be an improvement over the linear logistic.

II. Results

MSI at six microsatellite loci was determined by SP-PCR in PBL DNA from 17 normal blood bank donors. These individuals varied in age from 20 to 67 y/o. MSI has been shown to increase with allele size (Zhang et al., 1994). Therefore, range of sizes of the amplified products of the progenitor alleles in the individuals in the study was determined. The ranges were narrow (BAT26, 113 bp; D2S123, 152-172 bp; D5S346, 154-169 bp; D17S250, 134-153 bp; DMPK, 65-113 bp) and the different sized alleles were distributed evenly between members of the different age groups (data not shown), suggesting that any differences in MSI between older and younger individuals could not be attributed to allele size.

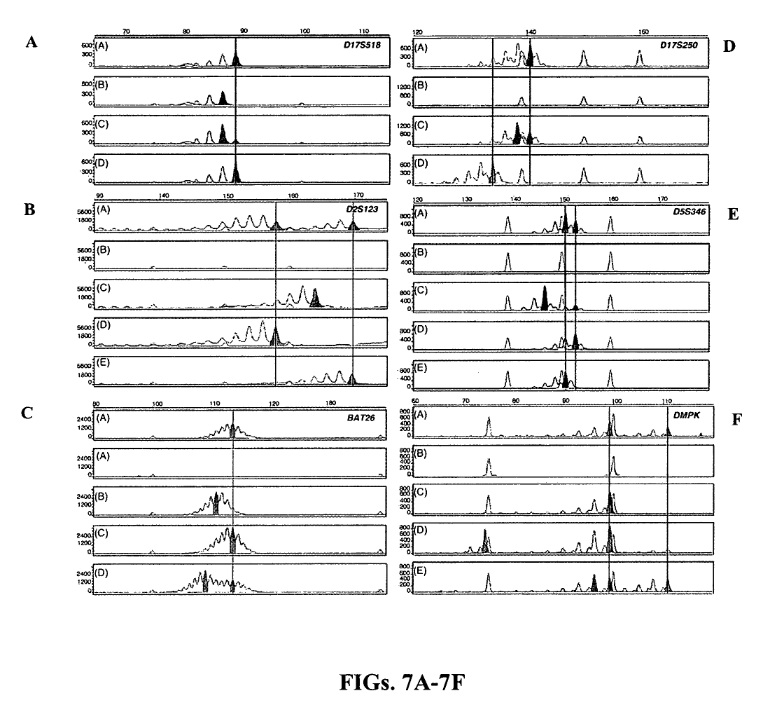

SP-PCR analysis of test microsatellite loci in PBLs of normal individuals. Examples chromatograms of the six microsatellite loci studied by SP-PCR are shown in FIG. 7. In these studies it was estimated that each small pool have a genome equivalency of 0.75. Therefore, the expectation was that in a series of chromatograms (each from the PCR products of a single pool) of any one locus there would be 0, 1, 2 and less frequently 3, or possibly 4 PCR fragments. For loci in individuals genotyped as being heterozygous, one would expect to see the separation of the progenitor alleles into different pools—panels D and E for D2S123; panels A and D for D17S250; panels D and E for D5S346.

Sometimes, mutant fragments were in the same pool as a progenitor—D17S518 panel C; BAT26 panel E; D17S250 panel C; D5S346 panel C; DMPK panels D and E. Since mutations were often single repeat unit deletions, they could be hidden in the stutter bands of the progenitor fragments when in the same pool. With the exception D17S518, this was particularly a problem with most dinucleotide repeats since the first stutter peaks were usually greater in height and area than the progenitor fragments. To identify if there was a mutant fragment in reaction, several normal control heterozygotes, where progenitor fragments were only 1 repeat unit apart (e.g., panelA of D5S346), were examined and indicated that the smaller fragments were stutter if their peak areas were less than 150% of the larger fragment (data not shown). This is referred to as the “rule of 150.” Here, since the smaller fragment in panel A of D5S346 had a peak area of >150% of the next larger fragment, it was not just stutter but the product of a second allele—in this case the second progenitor allele of this heterozygote. Similarly, the smaller fragment of D17S250 panel C contained a mutant fragment in addition to stutter. By the same token, the prominent stutters seen for both progenitor and mutant alleles of D2S123 were called as just that—stutter bands. This was not a problem for the trinucleotide DMPK or the dinucleotide D17S518 because stutter bands did not exceed the heights of progenitors. Consequently, when the smaller fragments exceeded the height of the one-repeat larger progenitor fragments at those loci, they were considered mutants—D17S518 panel C and DMPK panel E.

BAT26, typical of mononucleotide repeats, produced a Gaussian like distribution of peaks around its homozygous allele. If a mutant fragment was present in the same well as a progenitor, the inventors were not able to recognize it unless the main peak of the mutant fragment was at least three nucleotides separate from the progenitor. This leads to an underestimation of mutant alleles at this locus and is discussed further below.

Often mutants were captured into a pools devoid of progenitor fragments—D17S518 panel B; D2S123 panel C; BAT26 panel C. There were no problems evaluating them as mutants as there were similarly no problems in evaluating fragments separated from progenitor fragments by more than one repeat unit—D2S123 panel C; BAT26 panel E; D5S346 panel C; DMPK panel D.

Frequency of mutant fragments in different age groups. Table 10 summarizes all of the data over all the microsatellite loci studied in all the subjects in the different age groups. There, for all subjects are indicated the ages, genders, total calculated number of alleles screened and total number of mutants obtained at each locus. The mean MSI frequencies over all six loci were low (<0.01-0.047) in the PBLs of these normal individuals. However, the mean mutant frequencies over all of the individuals in each of the three age groups (0.009±0.005 in the 20-30 y/o; 0.019±0.007 in 35-50 y/o; 0.035±0.009 in 60-70 y/o) were significantly different from each other (p<0.01 by analyses of variants, see summaries under “Mean 6 loci” in Table 10).

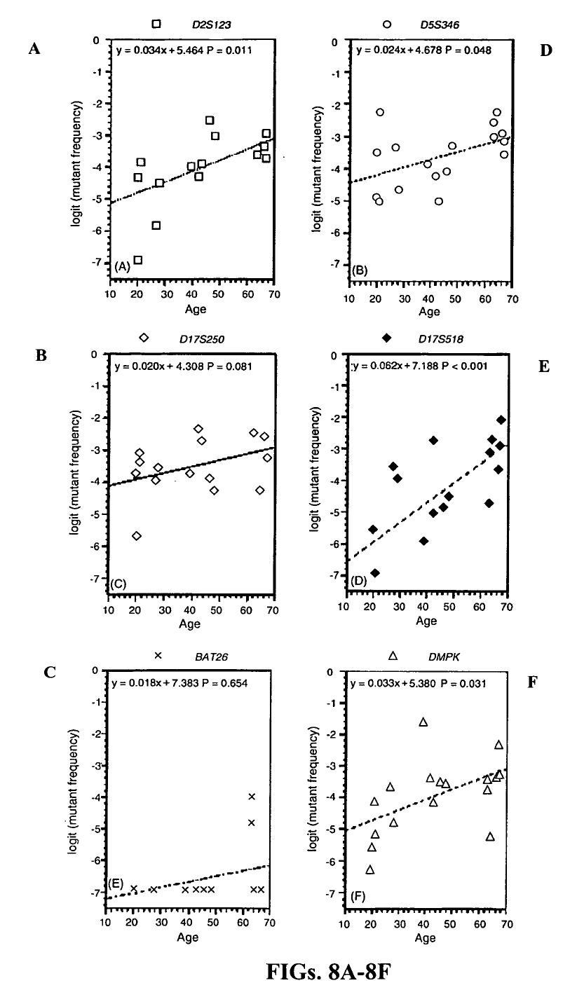

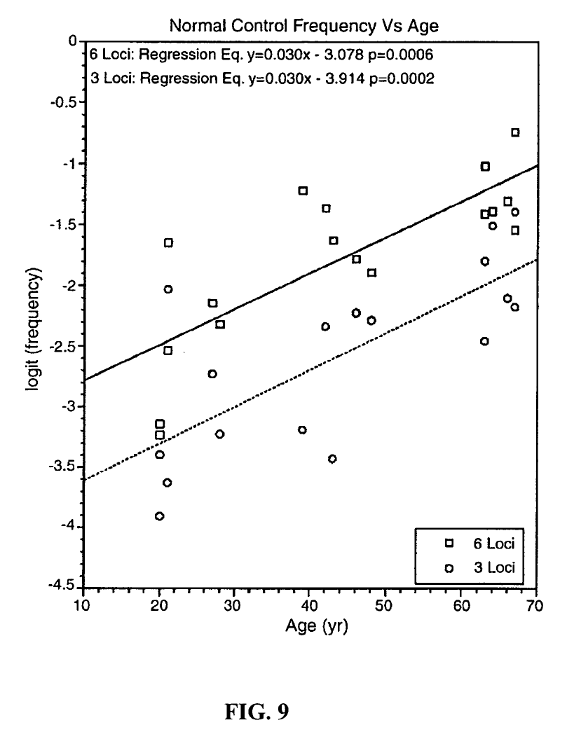

Linear regression analyses of Logit of the mutant frequencies plotted against age for each of the six loci are exhibited in FIG. 8. The p-values for the linear regressions, which tested the null hypothesis that frequency does not change with age, were significant (p<0.05) for all loci except for D17S250 (marginally significant at p=0.08) and BAT26 (minimally informative as discussed below). However, linear regression when data from all loci were plotted against age (FIG. 9) is highly significant (p=0.0006).

Therefore, in a multiple locus analysis in which the frequency of mutant fragments at each locus can be observed and calculated, the frequency of mutant fragments increases with age linearly in the PBLs of normal individuals.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. Aspects of one embodiment may be applied to other embodiments and vice versa. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### APPENDIX A

### A Demonstration that Alleles in a Well are Distributed as Independent Poisson Variates

An amount D of DNA is amplified in a well. The number of alleles in the well fit a Poisson distribution with mean cD, where c is the calibration constant. The probability of n alleles in a well is

$$\frac{{( cD )}^{n}}{n !} {ⅇ}^{- cD} \qquad ( 12 )$$

Suppose that there are three alleles labeled 1, 2, and 3. The frequencies of the alleles are, f₁, f₂, f₃, where the f's are positive and add to one. Let n₁, n₂, n₃ be three non-negative integers adding to n. Then given that there are n alleles in a well, the probability of n₁ of size 1, n₂ of size 2, and n₃ of size 3 is given by the multinomial distribution,

$$\frac{n !}{{n}_{1} ! {n}_{2} ! {n}_{3} !} {f}_{1}^{{n}_{1}} {f}_{2}^{{n}_{2}} {f}_{3}^{{n}_{3}}$$

We wish to show that the product of the two probabilities (12) and (13) is the same as the probability of (n₁, n₂, n₃) events from independent Poisson (13) distributions with means (cDf₁, cDf₂, cDf₃). The latter probability is

$$\frac{{( {cDf}_{1} )}^{{n}_{1}}}{{n}_{1} !} {ⅇ}^{- ( {cDf}_{1} )} \frac{{( {cDf}_{2} )}^{n 2}}{{n}_{2} !} {ⅇ}^{- ( {cDf}_{2} )} \frac{{( {cDf}_{3} )}^{{n}_{3}}}{{n}_{3} !} \qquad ( 14 )$$

The factorial terms are obviously the same in the two expressions as are the powers of fⁱ. Because f₁+f₂+f₃=1, it follows that
e^{-(cDf}^{₁}⁾+e^{-(cDf}^{₂}⁾+e^{-(cDf}^{₃}⁾=e^{-(cD)(f}^{₁}^{+f}^{₂}^{+f}^{₃}⁾=e^{-cD}
finishing the demonstration. The proof is the same for more than three alleles.

### REFERENCES

The following references, to the extent that they provide exemplary procedural or other details supplementary to those set forth herein, are specifically incorporated herein by reference.
Aaltonen et al., Science, 260:812-816, 1993.
Bacon et al., Nucleic Acids Res., 29:4405-4413, 2001.
Boland et al., Cancer Res., 58:5248-5257, 1998.
Botstein and Risch, Nat. Genet., 33 (Suppl):228-237, 2003.
Boyd, Prog. Clin. Biol. Res., 394:151-73, 1996.
Canzian et al., Cancer Res., 56:3331-3337, 1996.
Cleveland and Devlin, J. Am. Stat. Assoc., 83:596-610, 1988.
Coolbaugh-Murphy et al., Genomics, 84:419-430, 2004.
Coolbaugh-Murphy et al., Mech Ageing Dev., 126(10):1051-1059, 2005.
Fishel et al., Cell, 75:1027-1038, 1993.
Fonseca et al., Breast Can. Res., 7:R28-R32, 2005.
Fortune et al., Hum. Mol. Genet., 93:439-445, 2000.
Frazier et al., Cancer Res., 63(16):4805-4808, 2003.
Ionov et al., Nature, 363:558-561, 1993.
Jass, Int. J Colorectal Dis., 14:194-200, 1999.
Kane et al., Cancer Res., 57:808-811, 1997.
Kwok and Xiao, Hum. Mutat., 23:442-446, 2004.
Leeflang et al., Am. J Hum. Genet., 59:896-904, 1996.
Liehr, Endocrine Rev., 21:40-54, 2000.
Lynch, Gastroenterology, 104:1535, 1993.
Monckton and Jeffreys, Genomics, 11:465-467, 1991.
Monckton et al., Hum. Mol. Genet., 4:1-8, 1995.
Monckton et al., Nat. Genet., 15:193-196, 1997.
Parsons et al., Science, 268, 738-740, 1995.
Rechitsky et al., Mol. Cell Endocrinol., 183 Suppl 1: S65-68,2001.
Sia et al., Mol. Cellular Biol., 75:2851-2858, 1997.
Stuart and Ord, Kendall's Advanced Theory of Statistics, Oxford University Press, NY, 1991.
Thibodeau et al., Cancer Res., 58:1713-1718, 1998.
Tsao et al., Proc. Natl. Acad. Sci. USA, 97:1236-1241, 2000.
Wiesner et al., Proc. Natl. Acad. Sci. USA, 100:12961-12965, 2003.
Wong et al., Am. J. Hum. Genet., 56:114-122, 1995.
Yao et al., Proc. Natl. Acad. Sci. USA, 96:6850-6855, 1999.
Zhang et al., Nat. Genet., 7:531-535, 1994.
Zheng et al., Environ. Molecular Mutagenesis, 36:134-145, 2000.

What is claimed is:

1. A method for assessing an allele frequency in a DNA sample comprising the steps of:
(a) amplifying the DNA of the sample using amplification primers for at least one genetic marker; and
(b) calculating an allele frequency (fᵢ) of the amplified genetic markers.

2. The method of claim 1, wherein the allele frequency is determined by the formula:
$${\hat{f}}_{i} = \frac{{\hat{μ}}_{i}}{\hat{c}} ,$$
wherein \hat{μ}ᵢ is the maximum likelihood estimate of the mean number of allele i; and ĉ is the estimate of the calibration quantity.

3. The method of claim 1, further comprising assessing significance of the allele frequency within a sample or between two or more samples.

4. The method of claim 1, wherein prior to amplification the DNA is partitioned to less than 10 genome equivalents of DNA.

5. The method of claim 4, further comprising performing whole genome amplification on the DNA prior to partitioning.

6. The method of claim 4, wherein the DNA is partitioned to 0.5 to 2 genome equivalents.

7. The method of claim 1, wherein at least one allele is a mutant allele.

8. The method of claim 7, wherein the mutant allele frequency is less than 0.25.

9. The method of claim 8, wherein the mutant frequency is in the range of 0.01 to 0.25.

10. The method of claim 7, comprising determining the total mutant frequency.

11. The method of claim 10, wherein the total mutant frequency m is determined by the formula:
$$\hat{m} = \frac{{\sum }_{k} {\hat{μ}}_{k}}{{\sum }_{j} {\hat{μ}}_{j}}$$
where μ is the maximum likelihood estimate of the mean number of mutant alleles k and all alleles j; and j ranges over all alleles and k ranges over all mutant alleles.

12. The method of claim 1, wherein the genetic marker is a site specific marker, a multilocus marker, or a combination of site specific and multilocus markers.

13. The method of claim 12, wherein a genetic marker is a variable number tandem repeat (VNTR) marker, a minisatellite marker, a microsatellite marker, or a single nucleotide polymorphism (SNP) marker.

14. The method of claim 1, wherein the genetic marker is a microsatellite marker.

15. The method of claim 1, wherein the DNA is isolated from a cell, a tissue, a forensic sample, or a biological fluid.

16. The method of claim 15, wherein DNA is isolated from a blood sample, a buccal wash, a buccal swab, a vaginal swab, a histopathological sample, a skin sample, a skin scrape, sloughed skin, a biopsy, urine, saliva, semen, or a hair follicle.

17. The method of claim 1, wherein amplification is performed on 0.5 to 2 genome equivalents of DNA.

18. The method of claim 1, wherein amplification is performed on 3 to 12 pg of DNA.

19. The method of claim 1, wherein, the sample is from a subject that has, is suspected of having, or is at risk for developing cancer or a hyperproliferative condition.

20. The method of claim 19, wherein the subject is undergoing cancer therapy.

21. The method of claim 20, wherein the mutant frequency is correlated to development of resistance to a cancer therapy.

22. The method of claim 19, wherein the subject is a member of a family with a history of cancer.

23. The method of claim 19, wherein the subject has been exposed or is suspected of being exposed to genotoxic substance or environment.

24. The method of claim 19, further comprising correlating the allele frequencies of a mutant allele to a predisposition for cancer.

25. The method of claim 24, further comprising increasing monitoring of a subject for cancerous lesions or administering to the subject cancer preventative treatments.

26. A method of reconstructing the genotype of a subject comprising the steps of:
(a) obtaining DNA with an unknown genotype or haplotype;
(b) performing SP-PCR amplifying genetically linked markers in the DNA;
(c) partitioning the amplified DNA to single genome equivalents;
(d) conducting whole genome amplifications on the partitioned DNA; and
(e) assessing the phase of genetic marker by analysis of concordant amplification of genetically linked markers.

27. The method of claim 26, wherein the genetic marker is a site specific marker, a multilocus marker, or a combination of site specific and multilocus markers.

28. The method of claim 27, wherein a genetic marker is a variable number tandem repeat (VNTR) marker, a minisatellite marker, a microsatellite marker, or a single nucleotide polymorphism (SNP) marker.

29. The method of claim 26, wherein the DNA is isolated from a cell, a tissue, a forensic sample, or a biological fluid.

30. The method of claim 29, wherein DNA is isolated from a blood sample, a buccal wash, a buccal swab, a vaginal swab, a histopathological sample, a skin sample, a skin scrape, sloughed skin, a biopsy, urine, saliva, semen, or a hair follicle.

31. The method of claim 26, wherein amplification is performed on 0.5 to 2 genome equivalents of DNA.

32. The method of claim 31, wherein amplification is performed on 3 to 12 pg of DNA.

33. A method of genotyping a subject comprising the steps of:
(a) obtaining DNAwith an unknown genotype;
(b) diluting the DNA to obtain a DNA dilution comprising 0.5 to 2 genome equivalents of DNA and aliquoting the DNA into a number of small pools;
(c) conducting whole genome amplification on each pool;
(d) conducting a plurality of SP-PCR on each whole genome amplified pool amplifying a plurality of genetic markers;
(e) assessing the amplification of the genetic markers; and
(f) determining the linkage of the genetic markers to a trait or marker based on the assessment of the SP-PCR amplifications.

34. The method of claim 33, wherein assessing the genetic markers comprises:
(a) determining a maximum likelihood estimate of the mean number of alleles for a genetic marker in each amplification; and
(b) determining a frequency for each allele (allele frequency) across all amplifications for a DNA sample.

35. The method of claim 33, further comprising performing whole genome amplification on the DNA dilution of step (b) and using the amplified DNA for step (c).

36. The method of claim 35, wherein the whole genome amplification is performed on 0.5 to 2 genome equivalents of DNA.


SEQUENCE LISTING (22 sequences)

SEQ ID NO: 1 - DNA, 20 bp, Artificial Sequence
  Description of Artificial Sequence  Synthetic Primer
tcatggaggatgacgagttg

SEQ ID NO: 2 - DNA, 23 bp, Artificial Sequence
  Description of Artificial Sequence  Synthetic Primer
tggctctaaaatgctctgttctc

SEQ ID NO: 3 - DNA, 21 bp, Artificial Sequence
  Description of Artificial Sequence  Synthetic Primer
tcgcctccaagaatgtaagtg

SEQ ID NO: 4 - DNA, 21 bp, Artificial Sequence
  Description of Artificial Sequence  Synthetic Primer
gtttgaactgactacttttga

SEQ ID NO: 5 - DNA, 19 bp, Artificial Sequence
  Description of Artificial Sequence  Synthetic Primer
ccaatcaacatttttaacc

SEQ ID NO: 6 - DNA, 21 bp, Artificial Sequence
  Description of Artificial Sequence  Synthetic Primer
tgactacttttgacttcagcc

SEQ ID NO: 7 - DNA, 23 bp, Artificial Sequence
  Description of Artificial Sequence  Synthetic Primer
tgaccaaaagcatttctcttatg

SEQ ID NO: 8 - DNA, 27 bp, Artificial Sequence
  Description of Artificial Sequence  Synthetic Primer
cctttctgacttggataccatctatct

SEQ ID NO: 9 - DNA, 20 bp, Artificial Sequence
  Description of Artificial Sequence  Synthetic Primer
aaacaggatgcctgccttta

SEQ ID NO: 10 - DNA, 22 bp, Artificial Sequence
  Description of Artificial Sequence  Synthetic Primer
tgagaaatgaaatcgaatggag

SEQ ID NO: 11 - DNA, 23 bp, Artificial Sequence
  Description of Artificial Sequence  Synthetic Primer
tcagggaattgagagttacaggt

SEQ ID NO: 12 - DNA, 22 bp, Artificial Sequence
  Description of Artificial Sequence  Synthetic Primer
ggcctggttgtttccctagtat

SEQ ID NO: 13 - DNA, 20 bp, Artificial Sequence
  Description of Artificial Sequence  Synthetic Primer
aaggctgaggcaactgatgt

SEQ ID NO: 14 - DNA, 28 bp, Artificial Sequence
  Description of Artificial Sequence  Synthetic Primer
cacatacataaactttcaaatggtttca

SEQ ID NO: 15 - DNA, 20 bp, Artificial Sequence
  Description of Artificial Sequence  Synthetic Primer
tccgaaagtgctgggattac

SEQ ID NO: 16 - DNA, 19 bp, Artificial Sequence
  Description of Artificial Sequence  Synthetic Primer
tctccgcccagctccagtc

SEQ ID NO: 17 - DNA, 21 bp, Artificial Sequence
  Description of Artificial Sequence  Synthetic Primer
caggcctgcagtttgcccatc

SEQ ID NO: 18 - DNA, 21 bp, Artificial Sequence
  Description of Artificial Sequence  Synthetic Primer
aacggggctcgaagggtcctt

SEQ ID NO: 19 - DNA, 21 bp, Artificial Sequence
  Description of Artificial Sequence  Synthetic Primer
aaatggtctgtgatcccccca

SEQ ID NO: 20 - DNA, 26 bp, Artificial Sequence
  Description of Artificial Sequence  Synthetic Primer
tctttatagcattagtctctgggaca

SEQ ID NO: 21 - DNA, 20 bp, Artificial Sequence
  Description of Artificial Sequence  Synthetic Primer
tagtctctgggacacccaga

SEQ ID NO: 22 - DNA, 20 bp, Artificial Sequence
  Description of Artificial Sequence  Synthetic Primer
gatccagtggagactcagag